(19) United States
(12) Patent Application Publication
(10) Pub. No.: US 2024/0130654 A1
(43) Pub. Date: Apr. 25, 2024

(54) BIOMAGNETIC FIELD MEASUREMENT DEVICE, FLUX LOCKED LOOP UNIT, AND BIOMAGNETIC FIELD MEASUREMENT SYSTEM

(71) Applicants: Ricoh Company, Ltd., Tokyo (JP); KANAZAWA INSTITUTE OF TECHNOLOGY, Ishikawa (JP)

(72) Inventors: Takashi YASUI, Tokyo (JP); Ryoh MAEDA, Kanagawa (JP); Daisuke OYAMA, Ishikawa (JP); Yoshiaki ADACHI, Ishikawa (JP)

(21) Appl. No.: 18/461,782

(22) Filed: Sep. 5, 2023

(30) Foreign Application Priority Data
Oct. 19, 2022 (JP) 2022-168506

(51) Int. Cl. A61B 5/243; A61B 5/245

(52) U.S. Cl. CPC A61B 5/243; A61B 5/245; G01R 33/0356

(57) ABSTRACT

A biomagnetic field measurement device to measure a biomagnetic field includes a superconducting quantum interference device (SQUID) sensor, and includes a flux locked loop unit. The SQUID sensor includes an adjustment device configured to adjust a loop gain of the flux locked loop unit.

### CROSS-REFERENCE TO RELATED APPLICATIONS

The present application claims priority under 35 U.S.C. § 119 to Japanese Patent Application No. 2022-168506, filed Oct. 20, 2022, the contents of which are incorporated herein by reference in their entirety.

### BACKGROUND OF THE INVENTION

### 1. Field of the Invention

The present disclosure relates to a biomagnetic field measurement device, a flux locked loop (FLL) unit, and a biomagnetic field measurement system.

### 2. Description of the Related Art

Biomagnetic field measurement devices are known to measure biomagnetic fields using a superconducting quantum interference device (SQUID) that is a superconducting ring with a Josephson junction. In the measurement in the biomagnetic field using a SQUID sensor, a measurement characteristic is nonlinear.

With this arrangement, by using a flux locked loop (FLL) circuit to feed back a measured magnetic field to the SQUID sensor, the magnetic field is measured while maintaining a Φ-V characteristic, which is a characteristic of changes in a periodic voltage generated by the SQUID sensor, where the characteristic is near a lock point (linear region). As the FLL circuit, an analog FLL system and a digital FLL system are used. The analog FLL system is composed of only an analog circuit, and the digital FLL system composed of a circuit that first performs digital processing and then performs analog processing (see Patent Document 1 below).

In order to measure a magnetic field that is generated in a nerve that is weaker than a magnetic field generated by brain or heart activity, a biomagnetic field measurement system is known to generate an induced magnetic field by intentionally applying an electrical stimulation from electrodes that are attached to the skin of a living body, to thereby measure the generated induced magnetic field. In this type of biomagnetic field measurement system, for reducing magnetic field noise derived from the electrical stimulation, a fixing member capable of fixing a magnetic shielding cover to cover the electrodes may be provided in such a manner that the cover and the SQUID sensor have a certain relative positional relationship (see Patent Document 2 below).

### RELATED-ART DOCUMENT

### Patent Document

[Patent Document 1] Japanese Unexamined Patent Application Publication No. 2021-60396
[Patent Document 2] Japanese Unexamined Patent Application Publication No. 2017-15620

### Non-Patent Document

[Non-Patent Document 1] D. Drung, “High-Tc and low-Tc dc SQUID electronics,” Supercond. Sci. Technol. 16(2003), 1320-1336

### SUMMARY OF THE INVENTION

In one manner of the present disclosure, a biomagnetic field measurement device to measure a biomagnetic field includes a superconducting quantum interference device (SQUID) sensor, and includes a flux locked loop (FLL) unit. The FLL unit includes an adjustment device for adjusting a loop gain of the FLL unit.

### BRIEF DESCRIPTION OF THE DRAWINGS

FIG. 1 is a block diagram illustrating an example of a biomagnetic field measurement system including a biomagnetic field measurement device according to a first embodiment of the present disclosure;

FIG. 2 is a perspective diagram illustrating an example of a SQUID sensor array provided in a protruding portion of a dewar of FIG. 1;

FIG. 3 is a circuit block diagram illustrating an example of a digital FLL circuit provided on a FLL circuit unit of FIG. 1;

FIG. 4 is a diagram illustrating an example of a basic FLL circuit;

FIG. 5 is a diagram illustrating a closed loop characteristic of the basic FLL circuit;

FIG. 6 is a diagram illustrating an example of a basic digital FLL circuit;

FIG. 7 is a diagram illustrating an example of a closed-loop characteristic of the basic digital FLL circuit of FIG. 6;

FIG. 8 is a diagram illustrating examples of measurement locations for each measurement site of a biomagnetic field;

FIG. 9 is a diagram illustrating an example of a frequency-magnetic flux region and a slew rate in which the FLL circuit can operate;

FIG. 10 is a diagram illustrating an example of a measured waveform of the biomagnetic field and a waveform after artifact removal processing;

FIG. 11 is a diagram illustrating examples of a number of times of out-of-lock when the digital FLL circuit of FIG. 3 measures the biomagnetic field of a palm;

FIG. 12 is a diagram illustrating an example of a frequency characteristic before a loop gain is adjusted in the digital FLL circuit of FIG. 3;

FIG. 13 is a diagram illustrating an example of the frequency characteristic after the loop gain is adjusted in the digital FLL circuit of FIG. 3;

FIG. 14 is a circuit block diagram illustrating an example of a digital FLL circuit provided on the biomagnetic field measurement device according to a second embodiment of the present disclosure;

FIG. 15 is a diagram illustrating an example of frequency characteristic after the loop gain is adjusted in the digital FLL circuit of FIG. 14;

FIG. 16 is a circuit block diagram illustrating an example of an analog FLL circuit provided on the biomagnetic field measurement device according to a third embodiment of the present disclosure; and

FIG. 17 is a diagram illustrating an example of the closed-loop characteristic of the analog FLL circuit of FIG. 16.

### DESCRIPTION OF THE EMBODIMENTS

The inventors of this application have recognized the following related art information. When measuring an induced magnetic field generated in a nerve by an electrical stimulation that is applied to a living body, a stimulation artifact, which is an artifact caused by the electrical stimulation, is induced. If out-of-lock in which a Φ-V characteristic deviates from a location near a lock point is caused due to the stimulation artifact, a biomagnetic field may not be accurately measured. Conventionally, in order to suppress the occurrence of the out-of-lock, adjustment of electrical stimulation intensity and adjustment of a location at which the electrical stimulation is applied are performed. However, even if the adjustments are repeatedly performed, there are cases where the out-of-lock may not be sufficiently suppressed. Further, in a case where characteristics of each SQUID sensor varies, even if the electrodes for applying the electrical stimulation and a corresponding SQUID sensor have the same certain relative positional relationship, the intensity of the biomagnetic field to be measured varies due to variations in the characteristic of the SQUID, and there may be cases that the biomagnetic field cannot be accurately measured.

In view of the issues recognized by the inventors, the present disclosure has an object to accurately measure the biomagnetic field in a case where a SQUID sensor and a flux locked loop (FLL) unit are used to measure the biomagnetic field.

One or more embodiments will be described below with reference to the drawings. In the drawings, the same components are denoted by the same reference numerals, and redundant description may be omitted.

### First Embodiment

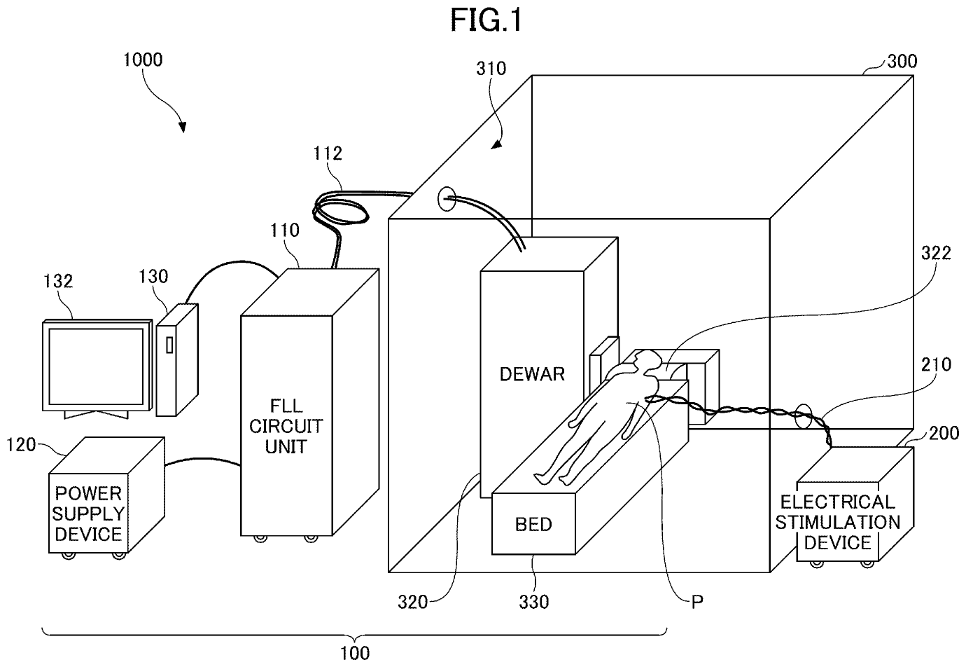

FIG. 1 is a block diagram illustrating an example of a biomagnetic field measurement system including a biomagnetic field measurement device according to a first embodiment of the present disclosure. For example, a biomagnetic field measurement system 1000 illustrated in FIG. 1 includes a biomagnetic field measurement device 100, an electrical stimulation device 200, and a magnetic shield room 300. The biomagnetic field measurement system 1000 also includes a dewar 320 (cooling container) and a bed 330 installed in the magnetic shield room 300.

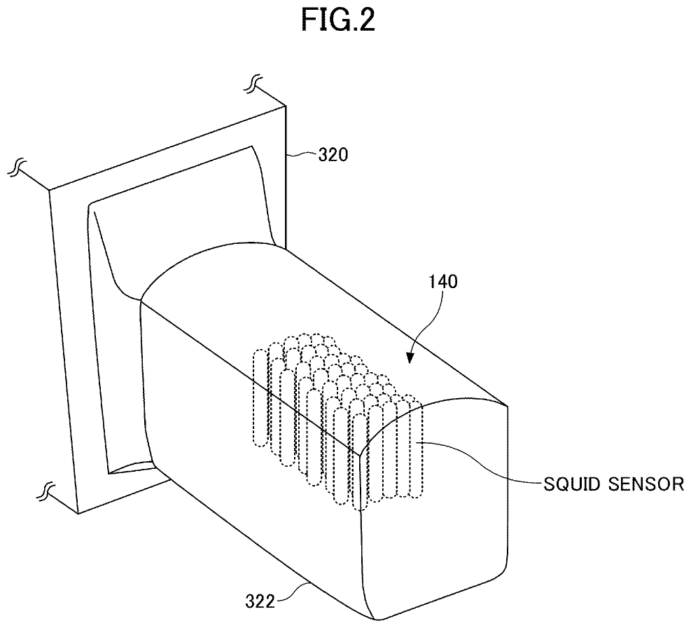

The biomagnetic field measurement system 1000 may include the biomagnetic field measurement device 100 used for measuring a biomagnetic field, the electrical stimulation device 200, and a SQUID sensor array 140 illustrated in FIG. 2, and may or may not include the magnetic shield room 300, the dewar 320, and the bed 330.

The biomagnetic field measurement device 100 includes a flux locked loop (FLL) circuit unit 110, a power supply device 120, a data processing device 130, and the SQUID sensor array 140 (not illustrated) disposed in a protruding portion 322 of the dewar 320. For example, the biomagnetic field measurement device 100 adopts a digital FLL system, and is used for a magnetospinograph. The biomagnetic field measurement device 100 can be applied to a magnetoencephalograph or a magnetocardiograph. The biomagnetic field measurement device 100 may also be applied to measurement of a nerve magnetic field or a muscle magnetic field.

TABLE 1

| | MAGNETO- | MAGNETO- | MAGNETO- |
|---|---|---|---|
| | SPINOGRAPH | CARDIO- | ENCEPHALO- |
| | (MSG) | GRAPH (MCG) | GRAPH (MEG) |
| MAGNETIC | Femtos to tens | tens of femtos | 10 f to 10 p |
| SENSITIVITY | of femtos | to 100 p | |
| (T) | | | |
| SIGNAL BAND | 100 to | O to 1k | 0(0.1) to |
| (HZ) | thousands | | hundreds |
| NUMBER OF | to 128 | 1 to 32 to 128 | 128 to 306 |
| CHANNELS | | | |

Table 1 illustrates an example of magnetic sensitivities (T), signal bands (Hz), and numbers of channels in measurement of the biomagnetic field signal for each application (measurement target). As illustrated in Table 1, the magnetic sensitivities, the signal bands, and the numbers of channels required for measurement of the biomagnetic field vary for the magnetospinograph (MSG), the magnetocardiograph (MCG), and the magnetoencephalograph (MEG). The channels vary in number depending on a measurement site and a measurement shape, and will likely further increase in number as the technique for analyzing a biomagnetic field signal becomes more advanced in the future.

In recent years, in addition to the magnetocardiograph and the magnetoencephalograph, application of the biomagnetic field measurement device to the magnetospinograph has become popular. The magnetospinograph measures an induced magnetic field generated in response to electrical stimulation input from outside. However, an artifact (noise) caused by the electrical stimulation affects a measurement result. Typically, the artifact is larger than the biomagnetic field.

The bed 330 is divided at a position corresponding to the neck when a subject P lies down. The protruding portion 322 of the dewar 320 is disposed in a gap between the divided beds 330. The dewar 320 is a cooling container that maintains an extremely low temperature environment using liquid helium, and the SQUID sensor array 140 and multiple feedback coils (not illustrated) are disposed inside the protruding portion 322 of the dewar 320.

A shield member 310 (wall member) that partitions the internal space and the external space of the magnetic shield room 300 is formed by stacking, for example, a plate member made of permalloy or the like that is a high magnetic permeability material and a plate member made of a conductor such as copper or aluminum. As illustrated in FIG. 1, the magnetic shield room 300 has, for example, a rectangular parallelepiped shape, and the shield members 310 are provided on four wall portions, a ceiling portion, and a floor portion. Note that the magnetic shield room 300 has a door that enables the transportation of various devices and entry and exit of people, but the door is omitted in FIG. 1.

The electrical stimulation device 200 is connected to the subject (living body) P via two signal cables 210 having a twisted cable structure that are passed through a through hole provided in one of the wall portions of the magnetic shield room 300. An electrode to be attached to the skin of the subject P is provided to the tip end of the signal cable 210. In the example illustrated in FIG. 1, the electrode is attached to the arm of the subject P. The electrical stimulation device 200 is connected to the data processing device 130 through a signal cable (not illustrated), and applies an electrical stimulation to the subject P in response to an instruction from the data processing device 130. The electrical stimulation device 200 is an example of an electrical stimulation input device.

In a case where a biomagnetic field is measured, an electrical stimulation is applied from the electrical stimulation device 200 to the subject P, and a biomagnetic field signal generated from the neck of the subject P induced by the electrical stimulation is detected by each SQUID sensor of the SQUID sensor array 140 (FIG. 2) arranged in the dewar 320 so as to face the neck. The biomagnetic field signal detected by each SQUID sensor is output as an electric signal from the inside of the dewar 320 to the FLL circuit unit 110 via signal cables 112 passed through another through hole provided in one of the wall portions of the magnetic shield room 300.

The FLL circuit unit 110 is connected to the power supply device 120 and the data processing device 130. For example, the data processing device 130 is a computer such as a PC or a server. The power supply device 120 is insulated from a commercial power supply in order to protect the subject P from electric shock. The FLL circuit unit 110 receives power from the power supply device 120, and operates based on an instruction from the data processing device 130. The FLL circuit unit 110 processes electrical signals indicating the biomagnetic field received from the SQUID sensors into digital signals by processing the electrical signals. The converted digital signals are sent to the data processing device 130, and accumulated as biomagnetic field data. A waveform or the like of the biomagnetic field data is then displayed on a display device 132 of the data processing device 130.

FIG. 2 is a perspective diagram illustrating an example of the SQUID sensor array 140 provided in the protruding portion 322 of the dewar 320 of FIG. 1. The dewar 320 is a cooling container (housing) for cooling the SQUID sensor array 140, and has a predetermined thickness for heat insulation. The SQUID sensors included in the SQUID sensor array 140 have, for example, a cylindrical shape extending in the vertical direction, and are arranged in a staggered manner in a top view. The tip end of each SQUID sensor is arranged along the inner surface of the convex spherical surface of the upper portion of the protruding portion 322. With this arrangement, the tip end of the SQUID sensor can be arranged, for example, according to the curved shape of the measurement target portion (the neck in FIG. 1) of the subject P lying on the bed 330, and the SQUID sensor can be opposed to the measurement target portion.

For example, the SQUID sensor array 140 has 44 SQUID sensors that function as a three-axis sensor. Each SQUID sensor has a gradiometer detection coil having an X-axis, a Y-axis, and a Z-axis, and an integrated chip for controlling the SQUID sensor, and a set of multiple signal lines for control and another set of multiple signal lines for taking out a magnetic field signal are wired therein. Note that each SQUID sensor may be a two-axis sensor having an X-axis and a Y-axis capable of measuring a magnetic field signal as a two-dimensional vector quantity, or a one axis sensor having only a Z-axis.

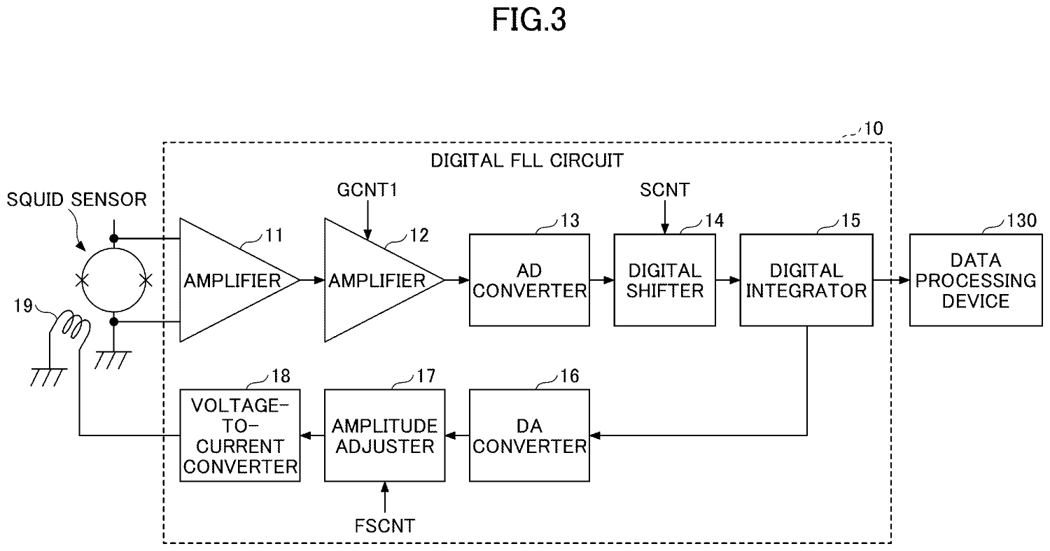

FIG. 3 is a circuit block diagram illustrating an example of a digital FLL circuit provided on the FLL circuit unit 110 illustrated in FIG. 1. A digital FLL circuit 10 illustrated in FIG. 3 is provided for each axis (each channel) of the SQUID sensor illustrated in FIG. 2. The digital FLL circuit 10 is an example of an FLL unit, and the SQUID sensor and the digital FLL circuit 10 are examples of a measurement device. Although not particularly limited, the FLL circuit unit 110 of FIG. 1 includes multiple control boards, and multiple digital FLL circuits 10 are implemented by each control board.

The SQUID sensor is a highly sensitive magnetic sensor that detects a magnetic field (magnetic flux) that is generated from a living body and that passes through a superconducting ring having a Josephson junction. For example, a SQUID sensor is configured by providing Josephson junctions at two locations (indicated by X marks) of the superconducting ring.

The SQUID sensor generates voltage that varies periodically with changes in the magnetic flux passing through the superconducting ring. Therefore, the magnetic flux passing through the superconducting ring can be obtained by measuring the voltage between both ends of the superconducting ring in a state in which the bias current is applied to the superconducting ring. Hereinafter, the characteristic of the periodic voltage change generated by the SQUID sensor is also referred to as a Φ-V characteristic.

The digital FLL circuit 10 includes an amplifier 11, an amplifier 12, an analog-to-digital (AD) converter 13, a digital shifter 14, a digital integrator 15, a digital-to-analog (DA) converter 16, an amplitude adjuster 17, a voltage-to-current converter 18, and a feedback coil 19. Although the feedback coil 19 arranged close to the SQUID sensor is physically separated from the digital FLL circuit 10, it may be included in a functional block of the digital FLL circuit 10.

The digital shifter 14 and the digital integrator 15 are implemented by, for example, a field-programmable gate array (FPGA) provided on the control board of the FLL circuit unit 110. The FPGA may be provided to handle the multiple digital FLL circuits 10 provided on the control board. For example, in a case where 16 digital FLL circuits 10 (for 16 channels) are provided on the control board, the FPGA includes the digital shifters 14 and the digital integrators 15 for the 16 channels.

Instead of the FPGA, an application specific integrated circuit (ASIC), an individual logic component, a general-purpose digital signal processor (DSP), or the like may be provided on the control board of the FLL circuit unit 110. In such a case, the digital shifter 14 and the digital integrator 15 may be implemented by the ASIC, the logic component, the general-purpose DSP, or the like.

The amplifier 11 whose inputs are connected to the both ends of the superconducting ring of the SQUID sensor amplifies an output voltage generated by the SQUID sensor in response to the strength of the magnetic field due to the magnetic flux passing through the SQUID sensor at a fixed amplification factor, and then outputs the amplified voltage to the amplifier 12 as a voltage signal. The amplifier 12 is able to set an amplification factor to any one of multiple amplification factors in accordance with a gain adjustment value GCNT1, amplifies the voltage received from the amplifier 11 at the set amplification factor, and outputs the amplified voltage to the AD converter 13 as a voltage signal. Note that the amplifier 12 can set a variable amplification factor at a sufficiently small value as compared with the amplifier 11 that performs amplification at a fixed amplification factor.

The AD converter 13 samples the voltage output from the amplifier 12 at a predetermined sampling frequency, and converts the sampled voltage into a digital value (a digital signal indicating a voltage magnitude). That is, the AD converter 13 converts the voltage output from the SQUID sensor according to the change in the magnetic field into a digital value. The AD converter 13 outputs the digital value generated by the conversion to the digital shifter 14.

The digital shifter 14 can set a shift amount to any one of multiple shift amounts in accordance with a shift adjustment value SCNT. The digital shifter 14 shifts, in accordance with the set shift adjustment value SCNT, the digital value (for example, little endian) from the AD converter 13 to the left or right by a designated number of bits.

The digital value is doubled by a 1-bit left shift, quadrupled by a 2-bit left shift, ½ times by a 1-bit right shift, and ¼ times by a 2-bit right shift. The digital shifter 14 outputs the shifted digital value to the digital integrator 15. Note that the shift adjustment value SCNT may include an instruction not to shift the digital value. In such a case, the digital shifter 14 outputs the digital value from the AD converter 13 to the digital integrator 15 as it is.

The digital integrator 15 receives, from the digital shifter 14, a signal value of a digital signal indicating a voltage magnitude that is obtained by amplifying the voltage of the SQUID sensor with respect to a voltage magnitude at a lock point (or an operating point) that is a starting point of counting of a magnetic flux quantum Φ0. The digital integrator 15 integrates a change in the received signal value (voltage magnitude), and outputs integrated data generated by the integration to the DA converter 16. The digital integrator 15 also outputs the integrated data to the data processing device 130.

The data processing device 130 stores, for example, the integrated data generated by the digital integrator 15 in a storage device (not illustrated) as digital data. The data processing device 130 generates image data, waveform data, and the like using the digital data stored in the storage device, and displays an image, a waveform, and the like represented by the generated image data, waveform data, and the like on a display device (not illustrated).

The DA converter 16 converts the signal waveform data, which is the integrated data integrated by the digital integrator 15, into voltage, and outputs the converted voltage to the amplitude adjuster 17 as a voltage signal. The DA converter 16 converts the digital value represented in a DA conversion bit length supplied from the digital integrator 15 into voltage (analog signal) with a preset full swing amplitude.

The amplitude adjuster 17 adjusts the amplification factor by converting the full scale (full swing amplitude) of the voltage output from the DA convertor 16 into any one of multiple full scales in accordance with a full-scale adjustment value FSCNT. The amplitude adjuster 17 converts the voltage magnitude from the DA converter 16 in accordance with the converted full scale, and outputs the converted voltage to the voltage-to-current converter 18.

Normally, the maximum value of the full swing amplitude of the DA converter 16 is often the power supply voltage amplitude value. For example, the full-scale adjustment value FSCNT indicates a magnification equal to or less than “1,” and the amplitude adjuster 17 adjusts the maximum full-swing amplitude (power supply voltage amplitude) to be equal to or less than 1.

The voltage-to-current converter 18 includes a resistance element that converts the voltage (voltage signal) whose full scale has been adjusted by the amplitude adjuster 17 into current (current signal), and outputs the converted current to the feedback coil 19.

The feedback coil 19 generates a magnetic field by the current signal that is received from the voltage-to-current converter 18, and feeds back the generated magnetic field to the SQUID sensor. With this arrangement, the voltage generated by the SQUID sensor can be maintained in the vicinity of the lock point (linear region) of the Φ-V characteristic, and the biomagnetic field signal can be measured with high accuracy.

The operations of the amplifier 12, the digital shifter 14, and the amplitude adjuster 17 are different from each other, however, the magnitude of the loop gain of the digital FLL circuit 10 can be adjusted as a whole. The actual loop gain of the digital FLL circuit 10 is represented by the product of the fixed amplification factor of the amplifier 11, the variable amplification factor of the amplifier 12, the variable magnification of the digital shifter 14, and the variable magnification of the amplitude adjuster 17.

By combining the variable amplification factor of the amplifier 12, the variable magnification of the digital shifter 14, and the variable magnification of the amplitude adjuster 17, the loop gain can be adjusted more finely than when it is adjusted by a single amplification factor or a single magnification. The amplifier 12, the digital shifter 14, and the amplitude adjuster 17 are examples of an adjuster that adjusts the loop gain of the digital FLL circuit 10 according to an adjustment value that can be set from outside.

Note that the gain adjustment value GCNT1, the shift adjustment value SCNT, and the full-scale adjustment value FSCNT are stored, for example, in a storage unit such as a register implemented by the FPGA together with the digital shifter 14 and the digital integrator 15. For example, the gain adjustment value GCNT1, the shift adjustment value SCNT, and the full-scale adjustment value FSCNT may be stored in a storage unit in the FPGA from the data processing device 130 when the biomagnetic field measurement device 100 is powered on, reset, or the like.

The shift adjustment value SCNT is supplied from the storage unit in the FPGA to the digital shifter 14 via a signal line in the FPGA. The gain adjustment value GCNT1 is supplied from the storage unit in FPGA to the amplifier 12 via a signal line provided on the control board. The full-scale adjustment value FSCNT is supplied from the storage unit in FPGA to the amplitude adjuster 17 via the signal line provided on the control board.

For example, as illustrated in FIG. 2, in a case where the SQUID sensor array 140 has 44 SQUID sensors functioning as three axis sensors, the biomagnetic field measurement device 100 has 132 digital FLL circuits 10. In order to reduce the number of wires provided on the control board of the FLL circuit unit 110, a common gain adjustment value GCNT1 and a common full-scale adjustment value FSCNT may be supplied to each of the amplifier 12 and the amplitude adjuster 17 for every predetermined number of digital FLL circuits 10 on each control board.

In such a case, the common gain adjustment value GCNT1 and the common full-scale adjustment value FSCNT are preferably supplied to the digital FLL circuit 10 connected to the SQUID sensor having the same sensor characteristics. With this arrangement, the biomagnetic field measurement device 100 with less variation in frequency characteristic can be achieved.

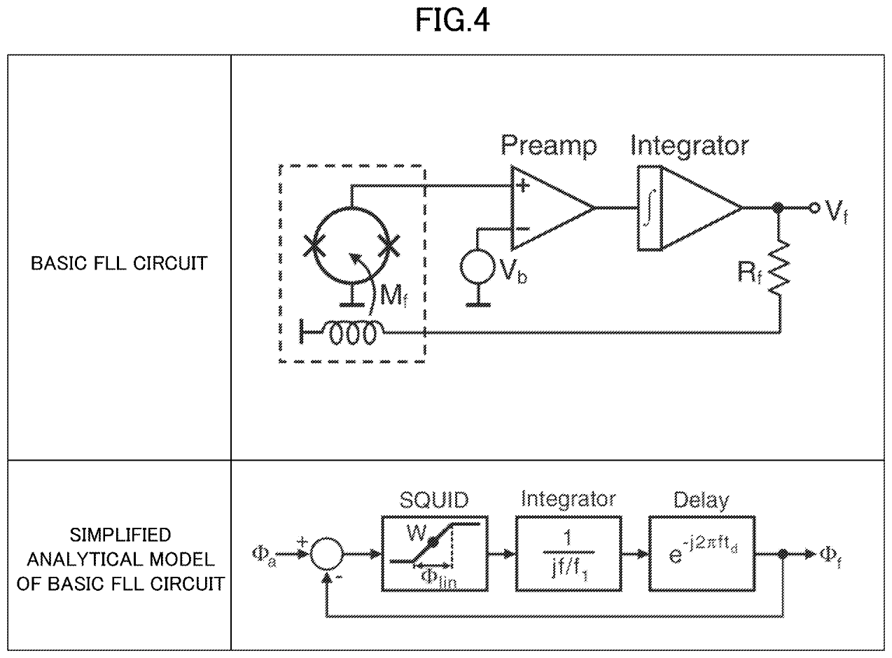

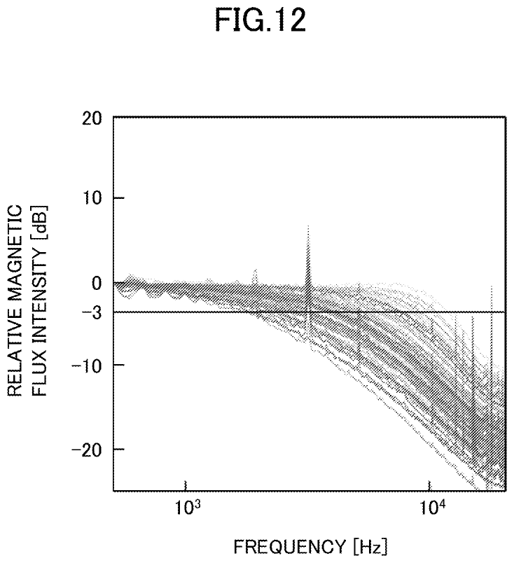

FIG. 4 is a diagram illustrating an example of a basic FLL circuit (for example, an analog FLL circuit). The basic FLL circuit illustrated and a simplified analytical model of the basic FLL circuit in FIG. 4 are described in FIG. 3 and FIG. 12 of the document “D. Drung, “High-Tc and low-Tc dc SQUID electronics”, Supercond. Sci. Technol. 16 (2003), 1320 to 1336”, respectively. In the analytical model of FIG. 4, a f₁ represents a unity gain frequency of the open loop (cutoff (−3 dB) frequency of the integrator), and a td represents a delay of the FLL loop.

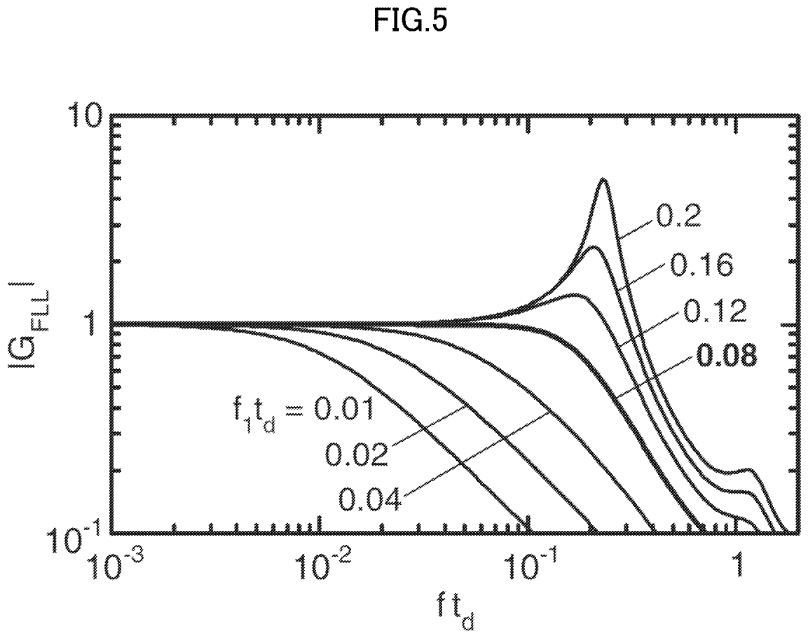

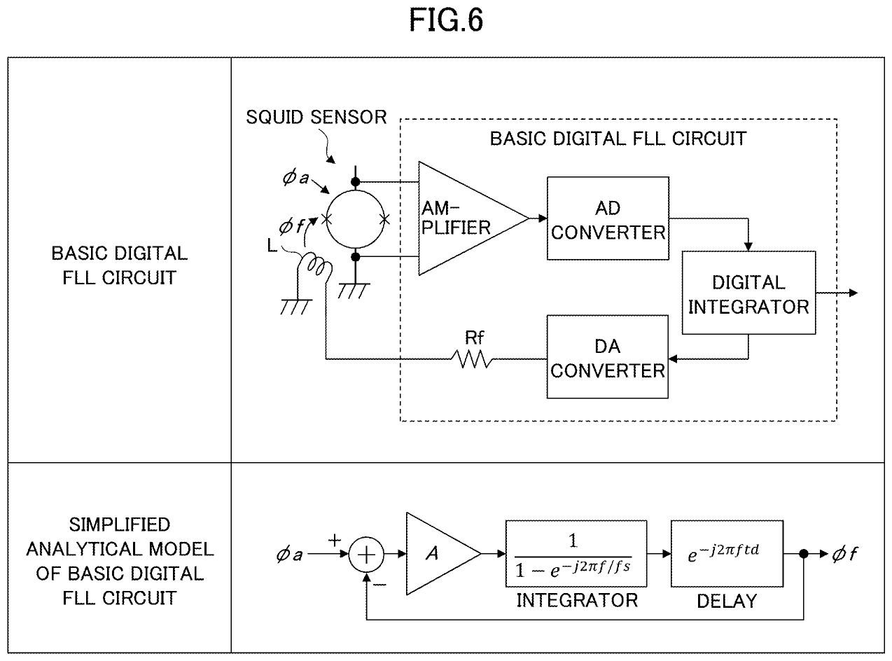

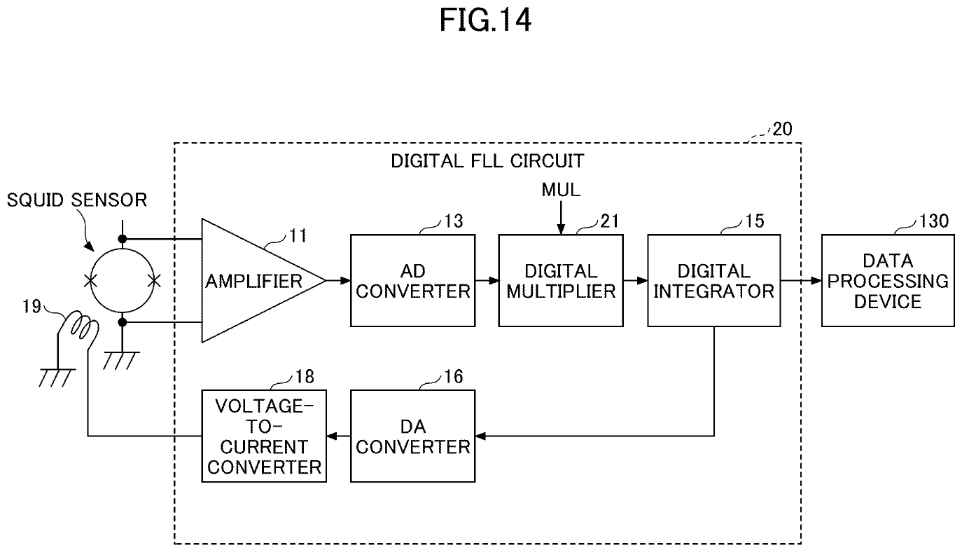

FIG. 5 illustrates a closed loop characteristic of the basic FLL circuit. The closed loop characteristic illustrated in FIG. 5 is described in FIG. 14 of the document “D. Drung, “High-Tc and low-Tc dc SQUID electronics”, Supercond. Sci. Technol. 16 (2003), pp. 1320 to 1336”. In the range of ft_{d}<0.08 (small f₁), the closed loop characteristic behaves like a first-order low-pass filter. In the range of ft_{d}>0.08 (large f₁), the closed loop characteristic causes a large phase delay, a peak appears, and the system becomes unstable. The closed loop characteristic illustrated in FIG. 5 corresponds to the closed loop characteristic when a value A is set to “1”, which will be described with reference to FIG. 6.

Therefore, the FLL circuit is preferably designed to behave like a first order low-pass filter in such a manner that the system does not become unstable in either the analog FLL circuit or the digital FLL circuit.

FIG. 6 is a circuit block diagram illustrating an example of the basic digital FLL circuit. The basic digital FLL circuit includes an amplifier, an analog-to-digital (AD) converter, a digital integrator, a digital-to-analog (DA) converter, a resistor Rf serving as a voltage-to-current converter, and a feedback coil L that are connected in a loop with respect to the SQUID sensor.

The basic digital FLL circuit includes the digital integrator, and thus DA conversion is also performed after once digitization in the analysis model. With this arrangement, the signal changes from a continuous system to a discrete system and once again to a continuous system. In the discrete system, a well-known approximation formula based on the Z-transform is used.

Here, the value A represents the loop gain of the entire digital FLL circuit, but is not merely the gain of the amplifier. The A value is a value in which all of a conversion ratio from the magnetic field signal to the electric signal, an eigenvalue of the SQUID sensor such as the maximum inclination of the Φ-V characteristic, a number of bits at the time of AD conversion and an input maximum amplitude setting, and a number of bits at the time of DA conversion and a magnification at the output maximum amplitude setting are taken into consideration.

In the digital FLL circuit 10 illustrated in FIG. 3, the value A is proportional to the fixed amplification factor of the amplifier 11 and the variable amplification factor of the amplifier 12. The value A is also inversely proportional to the number obtained by dividing the maximum amplitude setting of the AD converter 13 by a power of 2 of the number shifted by the digital shifter 14. The value A is also proportional to the value obtained by dividing the amplitude of the amplitude adjuster 17 for adjusting the full swing amplitude of the DA converter 16 by a power of 2 of the conversion bit number of the DA converter 16.

However, the loop gain need not be adjusted using all of the components, namely, the amplifier 12, the digital shifter 14, and the amplitude adjuster 17. The loop gain may be adjusted using one or two of the amplifier 12, the digital shifter 14 and the amplitude adjuster 17, depending on the adjustment range.

In a case where the loop gain is adjusted by one element, the digital FLL circuit 10 of FIG. 3 need not include a pair of the digital shifter 14 and the amplitude adjuster 17, a pair of the amplifier 12 and the amplitude adjuster 17, or a pair of the amplifier 12 and the digital shifter 14. In a case where the digital FLL circuit 10 does not include the pair of the digital shifter 14 and the amplitude adjuster 17, the output of the AD converter 13 is connected to the input of the digital integrator 15, and the output of the DA converter 16 is connected to the input of the voltage-to-current converter 18.

In a case where the digital FLL circuit 10 does not include the pair of the amplifier 12 and the amplitude adjuster 17, the output of the amplifier 11 is connected to the input of the AD converter 13, and the output of the DA converter 16 is connected to the input of the voltage-to-current converter 18. In a case where the digital FLL circuit 10 does not include the pair of the amplifier 12 and the digital shifter 14, the output of the amplifier 11 is connected to the input of the AD converter 13, and the output of the AD converter 13 is connected to the input of the digital integrator 15.

In a case where the loop gain is adjusted by two elements, the digital FLL circuit 10 of FIG. 3 need not include the amplitude adjuster 17, the digital shifter 14, or the amplifier 12. In a case where the digital FLL circuit 10 does not include the amplitude adjuster 17, the output of the DA converter 16 is connected to the input of the voltage-to-current converter 18. In a case where the digital FLL circuit 10 does not include the digital shifter 14, the output of the AD converter 13 is connected to the input of the digital integrator 15. In a case where the digital FLL circuit 10 does not include the amplifier 12, the output of the amplifier 11 is connected to the input of the AD converter 13.

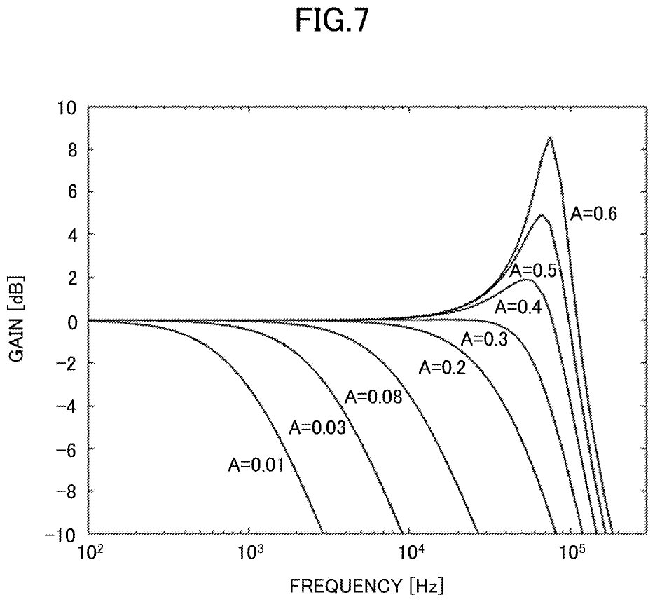

FIG. 7 is a diagram illustrating an example of the closed loop characteristic of the basic digital FLL circuit of FIG. 6. In FIG. 7, the magnitude of the output (output voltage) signal relative to the magnetic signal having the frequency indicated on the horizontal axis is indicated as a gain [dB] on the vertical axis. As for the closed loop characteristic of the basic digital FLL circuit, a waveform similar to the closed loop characteristic of the basic FLL circuit illustrated in FIG. 5 is obtained although the analysis model used is different from the analysis model illustrated in FIG. 4. For stability of the system, it is preferable that the system is designed to behave similarly to the first order low-pass filter. It can be seen from the closed loop characteristic illustrated in FIG. 7 that when the value A is equal to or less than 0.3, the frequency characteristic of the digital FLL circuit can be stably controlled in accordance with the value A.

In the digital FLL circuit 10 of FIG. 3, the A value (loop gain) can be adjusted for each channel of the SQUID sensor by one or more of the amplifier 12, the digital shifter 14, and the amplitude adjuster 17. For example, by appropriately adjusting the A value (loop gain) for each channel of the SQUID sensor in accordance with the characteristics of the individual SQUID sensors in the SQUID sensor array 140, the frequency characteristic of the digital FLL circuit 10 can be controlled and the biomagnetic field can be measured with high accuracy.

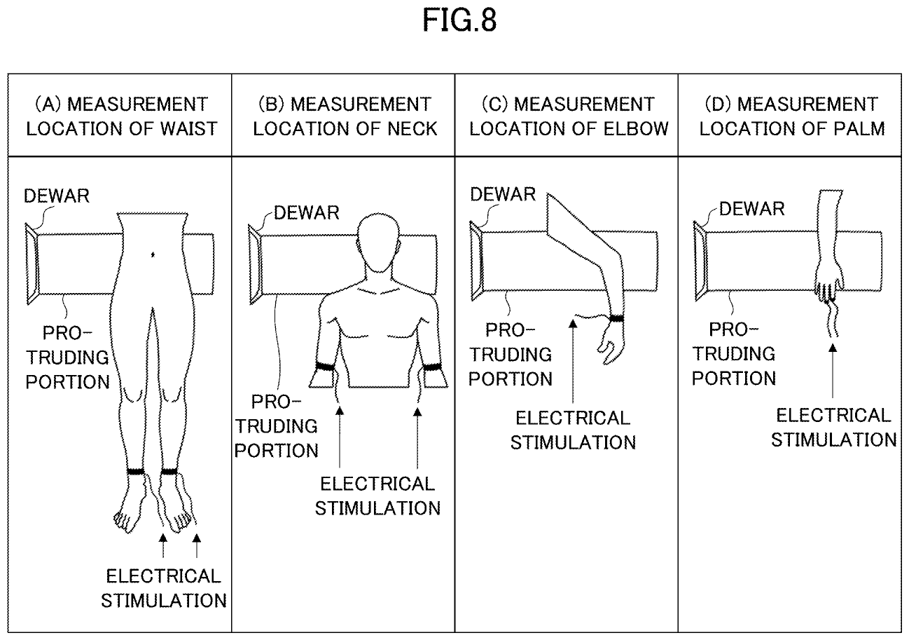

FIG. 8 is a diagram illustrating examples of measurement locations for each measurement site of the biomagnetic field. Representative examples of the measurement sites of the biomagnetic field include the waist and the neck centered on the central nervous system, and the elbow and the palm for measuring peripheral nerves. FIG. 8 illustrates examples of measurement locations when biomagnetic fields are measured at (A) the waist, (B) the neck, (C) the elbow, and (D) the palm.

In the example of the measurement location of the waist, the electrical stimulation is applied from the ankles. However, the electrical stimulation may be applied from below the knees or above the knees depending on the nerve conduction to be measured. The locations of the electrodes where the electrical stimulation is applied are illustrated in black. In a case where the biomagnetic field is measured on the palm, the electrical stimulation is applied to the tips of the index finger and middle finger. In a case where electrical stimulation is applied to two locations of the subject P with the electrical stimulation device 200 or the like in FIG. 1, the electrical stimulation can be selected to be applied to one by one or both at the same time.

It is generally known that the magnitude of a magnetic field is inversely proportional to the square of the distance from a magnetic field source (Coulomb's law). In a case where the electrical stimulation is applied to the subject P, the magnetic field signal includes not only the biomagnetic field signal but also the magnetic field signal induced by the electrical stimulation. The magnetic field signal induced by the electrical stimulation is referred to as an artifact or stimulation artifact. An artifact is artificially induced noise.

Due to the electro-magnetic interaction, the closer the location of the electrical stimulation is to the measurement site, the greater the strength of the detected magnetic field signal of the stimulation artifact. For example, the signal intensity of the stimulation artifact increases in the order of (A) the waist, (B) the neck, (C) the elbow, and (D) the palm. Furthermore, as the signal intensity of the stimulation artifact increases, for both the digital FLL circuit and the analog FLL circuit, the out-of-lock is more likely to occur. In general, the signal intensity of the stimulation artifact is larger than that of the biomagnetic field signal, and the frequency of the stimulation artifact includes a component higher than that of the biomagnetic field signal.

In the document “D. Drung, “High-Tc and low-Tc dc SQUID electronics”, Supercond. Sci. Technol. 16 (2003), 1320 to 1336”, a slew rate SR representing the rate of change of the magnetic flux signal over time is expressed by Equation (1). The symbol Φf in Equation (1) represents a feedback magnetic flux.

[Equation 1]

SR≡\dot{Φ}f=|∂Φf/∂t|max (1)

The relationship between the operable amplitude and frequency of the FLL circuit can be derived from Equation (1). An applied magnetic flux Φa is represented by Equation (2), where an amplitude of an applied magnetic flux signal at a certain frequency is denoted by Φp and the frequency is denoted by f.

[Equation 2]

Φa=Φp sin2πft (2)

Assuming that the applied magnetic flux Φa in Equation (2) and the feedback magnetic flux Φf in Equation (1) are in equilibrium, Equation (1) can be expressed by Equation (3).

$$\begin{matrix}[ Equation 3 ] & \\ SR = \frac{\partial Φ f}{\partial t} \vert max = 2 π f Φ p & ( 3 )\end{matrix}$$

Furthermore, an operable amplitude Φp at the frequency f is expressed by Equation (4), and is inversely proportional to the frequency f. Therefore, it can be seen that the operable magnetic flux intensity decreases as the frequency f increases.

[Equation 4]

φp=SR/2πf (4)

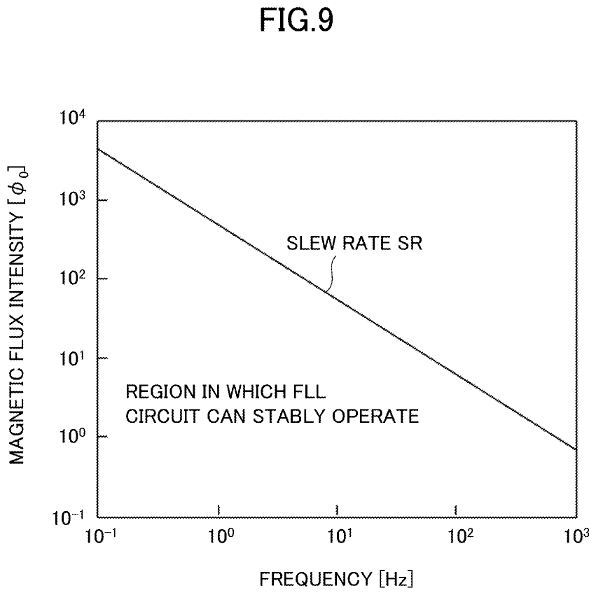

FIG. 9 is a diagram illustrating an example of a frequency-magnetic flux region in which the FLL circuit can operate and the slew rate SR. When the parameters of the applied signal are plotted with the frequency on the horizontal axis and the amplitude on the vertical axis, the region at the lower left of the straight line is a region in which the FLL circuit can stably operate, and this straight line coincides with the slew rate SR given by Equation (3).

In the measurement of the biomagnetic field of the nervous system, the location at which the electrical stimulation is applied and the intensity of the electrical stimulation are determined so that the nerve to be measured fires. For example, as illustrated in FIG. 8, when arranging in order of proximity from the location where the electrical stimulation is applied to the biomagnetic field measurement site, the order is (D) the palm, (C) the elbow, (B) the neck, and (A) the waist.

The location at which the electrical stimulation is applied and the intensity of the electrical stimulation are determined, for example, in consideration of the following three factors. (1) The distance from the location to be stimulated to the nerve to be measured is short and the nerve is easily fired, (2) a sufficiently measurable biomagnetic field is obtained when the nerve to be measured is fired, and (3) the FLL circuit neither exceeds the operation range nor causes the out-of-lock.

In the vicinity of a joint portion where the nerve to be measured is close to the surface of the skin, candidates for a location to be stimulated are relatively easily narrowed down. On the other hand, as illustrated in FIG. 9, as the frequency increases, the measurable magnetic flux intensity of the artifact that is much larger than that of the biomagnetic field signal and includes a high frequency component decreases. For this reason, it is very difficult to adjust the intensity and the application frequency of the electrical stimulation pulse, and in some cases, there is a possibility that measurement cannot be performed. There is also a problem in that even if measurement is possible, the biomagnetic field signal is very small and cannot be analyzed.

Therefore, in the present embodiment, in the measurement of the biomagnetic field with the electrical stimulation, the frequency characteristic of the FLL that has been conventionally fixed is controlled by using the same characteristic as the low-pass filter characteristic of the FLL instead of application criteria of the electrical stimulation. With this arrangement, the out-of-lock of the FLL circuit can be avoided by cutting a high-frequency component, so that a desired biomagnetic field signal can be obtained.

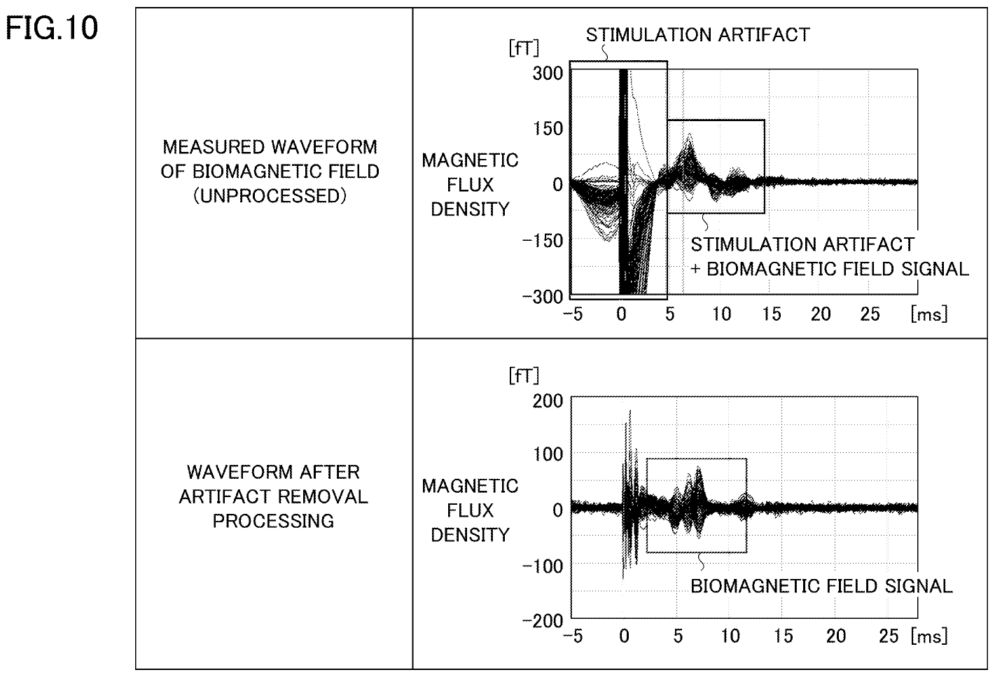

FIG. 10 is a diagram illustrating an example of a measured waveform of the biomagnetic field and a waveform after artifact removal processing. The waveform illustrated in FIG. 10 is a waveform of the magnetic field signal measured in the cervical spine (change in magnetic flux density over time), and data of all channels of the SQUID sensor array is simultaneously illustrated. In the measurement waveform (unprocessed) of the biomagnetic field, after a large-amplitude artifact (stimulation artifact) due to the electrical stimulation appears immediately after the start of measurement, a signal obtained by adding a biomagnetic field signal to the stimulation artifact appears.

In the waveform after the artifact removal processing with the dedicated processing software, a large stimulus artifact is removed, the out-of-lock of the digital FLL circuit has not occurred, and measurement is correctly performed. Then, a biomagnetic field signal containing almost no stimulation artifact is obtained.

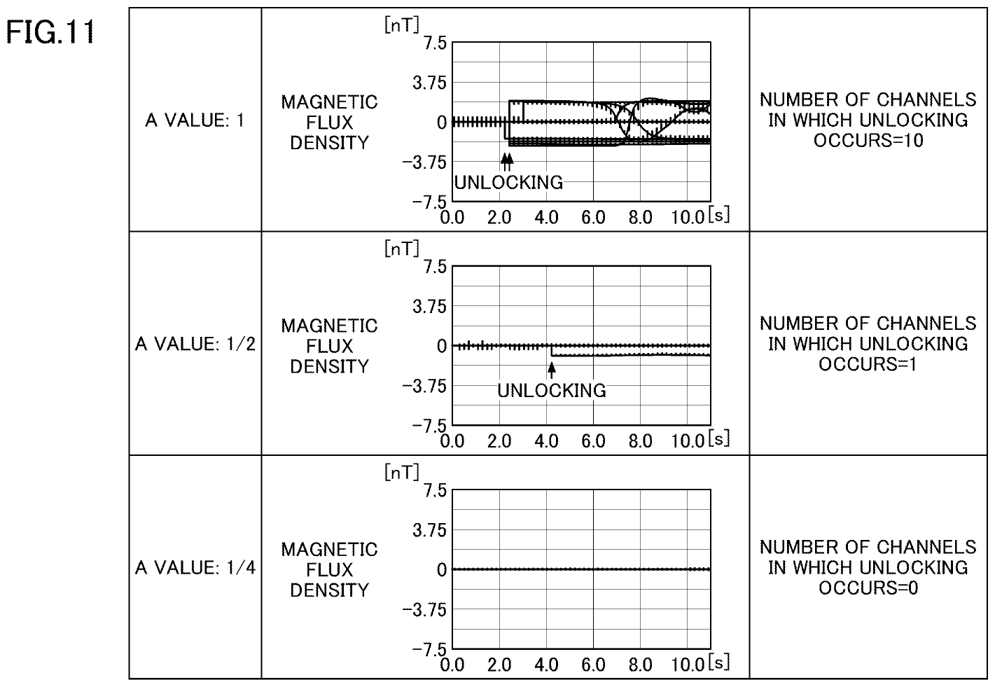

FIG. 11 is a diagram illustrating examples of a number of times of out-of-lock when the digital FLL circuit 10 of FIG. 3 measures the biomagnetic field of the palm. Similar to FIG. 10, FIG. 11 illustrates a change in magnetic flux density over time. The number of channels of the SQUID sensors included in the biomagnetic field measurement device 100 of FIG. 1 is 132 (44 SQUID sensors×3 axes). As illustrated in (D) of FIG. 8, the electrical stimulation to the palm is applied thorough the tips of the index finger and middle finger. The stimulation criteria are as follows: simultaneous stimulation for carpal tunnel, index finger and middle finger, the frequency: 5 Hz, the duration: 0.05 ms, and the intensity: 40 mA.

In order to observe the out-of-lock of the digital FLL circuit 10, the vertical axis is set to a nT order that is six orders of magnitude larger than a fT order used in the normal measurement of the biomagnetic field. Therefore, the biomagnetic field signal is compressed in the vertical axis direction and cannot be seen. Needle-like lines extending in the vertical axis direction in the waveform illustrated in FIG. 11 are the stimulation artifacts illustrated in FIG. 10. The digital FLL processes data using signed binary numbers, and thus the sign is changed when the out-of-lock occurs and overflow occurs, and the digital FLL may operate so as to move between the positive and negative maximum values. By this operation, the occurrence of the out-of-lock can be determined.

In order to observe the dependency of the value A, in the amplitude adjuster 17 of the digital FLL circuit 10 of FIG. 3, the ratio to the full scale is set to “1,” “½,” and “¼.” The value A is also set to “1,” “½,” and “¼,” and the number of times of out-of-lock is measured in each case. As the value A decreases to “1,” “½,” and “¼,” the number of channels in which out-of-lock occurs decreases to “10,” “1,” and “0,” respectively.

In FIG. 11, the value A is adjusted by controlling the amplitude by the amplitude adjuster 17, and thus the amplitude of the magnetic flux density due to the stimulation artifact also decreases as the value A decreases. Note that “1,” “½,” and “¼” of the A value may be adjusted using the amplifier 12 or the digital shifter 14 instead of the amplitude adjuster 17, or may be adjusted using two or three among the amplifier 12, the digital shifter 14, and the amplitude adjuster 17. By using multiple devices such as the amplifiers 12, digital shifters 14, and amplitude adjusters 17, the A value can be adjusted more finely.

In this embodiment, in a case where the induced magnetic field generated by applying the electrical stimulation to the subject P is measured, the loop gain can be adjusted for each channel (that is, for each digital FLL circuit 10). Therefore, in the biomagnetic field measurement device 100 including a large number of channels, the out-of-lock of the digital FLL circuits 10 can be avoided, and the frequency characteristic can be made uniform among the digital FLL circuits 10. As a result, the biomagnetic field measurement device 100 can be stably operated, and the biomagnetic field can be measured with high accuracy.

The loop gain can also be adjusted for each channel, and thus variations in characteristics among the channels of the SQUID sensor can be allowed to some extent. As a result, the SQUID sensor that has been conventionally treated as a defective product because the sensor characteristics are out of standard can be saved. Thus, the manufacturing yield of the SQUID sensor can be improved, and the manufacturing cost of the SQUID sensor and the biomagnetic field measurement device 100 can be reduced.

In a case where the variation in sensor characteristics among the channels is small, the loop gain may be adjusted not for each channel but for each group of the channels. This makes it possible to use the gain adjustment value GCNT1, the shift adjustment value SCNT, and the full-scale adjustment value FSCNT common to each group of the channels. As a result, the capacity of the storage unit that holds the gain adjustment value GCNT1, the shift adjustment value SCNT, and the full-scale adjustment value FSCNT can be reduced, and the circuit scale of the digital FLL circuit 10 can be reduced.

FIG. 12 is a diagram illustrating an example of the frequency characteristic before the loop gain is adjusted in the digital FLL circuit 10 of FIG. 3. The frequency characteristic illustrated in FIG. 12 is obtained by using 76 SQUID sensors, and using a loop gain of the same value, without adjusting the loop gain of each channel. Each curve illustrated in FIG. 12 represents the frequency characteristic of one channel.

The frequency characteristic of the FLL circuit can be evaluated by measuring the noise characteristic of the system in a state where no magnetic field input is applied to the SQUID sensor installed in the magnetic shield room 300. The noise measured in the state where no magnetic field input is applied to the SQUID sensor includes environmental noise such as geomagnetism or vibration, but mainly includes noise of the SQUID sensor itself, thermal noise of the FLL circuit and the dewar 320, and the like. It is difficult to separate the noise components.

However, for example, it is known that an amplifier generates 1/f noise seen in a low band of a frequency region and noise that is constant with respect to a frequency in a wide band seen in a high band. Therefore, the frequency characteristic in a region higher than the 1/f noise of the FLL circuit can be examined by using the broadband noise generated in the amplifier, and a cutoff frequency of the FLL circuit can be measured.

The noise characteristic and the characteristic of the SQUID sensor vary for each channel (for each axis of the SQUID sensor). In the frequency characteristic illustrated in FIG. 12, a specific frequency of a specific channel is set as a reference (0 dB), and the cutoff frequency of the digital FLL circuit 10 is set as −3 dB and illustrated as a line parallel to the horizontal axis (frequency axis). In FIG. 12, it can be seen that the frequency range at which the curves of the frequency characteristic of each channel intersect with the line of the cutoff frequency is wide, and the cutoff frequency varies.

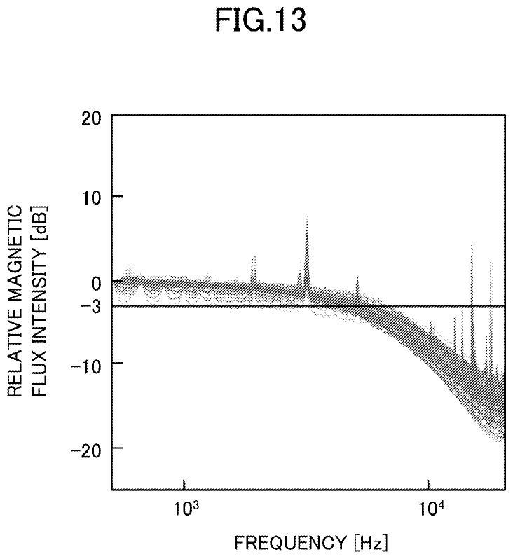

FIG. 13 is a diagram illustrating an example of the frequency characteristic after the loop gain is adjusted in the digital FLL circuit 10 of FIG. 3. Detailed description of elements substantially the same as those in FIG. 12 will be omitted.

The method of acquiring the frequency characteristic illustrated in FIG. 13 is the same as the method of acquiring the frequency characteristic illustrated in FIG. 12 except that the loop gain is adjusted for each group of a predetermined number of channels. For example, the A value (that is, loop gain) of the digital FLL circuit 10 is adjusted by fixing the adjustment value of the full swing amplitude of the amplitude adjuster 17 in FIG. 3 and changing the amplification factor of the amplifier 12 and the magnification (shift amount) of the digital shifter 14.

In consideration of the stability of the digital FLL circuit 10, the amplification factor of the amplifier 12 can be set to one of two types, and the magnification of the digital shifter 14 can be set to one of three types. Groups are then generated by collecting the channels whose frequency characteristics are close to each other in FIG. 12. For each group, the amplification factor of the amplifier 12 is set to either one of two types, and the magnification of the digital shifter 14 is set to either one of three types. With this arrangement, the frequency characteristic of FIG. 13 is obtained.

In FIG. 13, it can be seen that the frequency range at which the curves of the frequency characteristic of each channel intersect the line of the cutoff frequency (−3 dB) is narrower than that in FIG. 12, and the variation in the cutoff frequency is reduced. Because the variation in the cutoff frequency can be suppressed, the occurrence of the out-of-lock can be suppressed, and a decrease in the measurement accuracy of the biomagnetic field can also be suppressed. Furthermore, as described above, the SQUID sensor that has been treated as a defective product can be saved, and the manufacturing cost of the SQUID sensor and the biomagnetic field measurement device 100 can be reduced.

As described above, in the first embodiment, the loop gain of the digital FLL circuit 10 can be adjusted for each channel by making one or more characteristics of the amplifier 12, the digital shifter 14, and the amplitude adjuster 17 changeable in accordance with an adjustment value that can be set from outside. For example, the loop gain can be adjusted more finely using two or three among the amplifier 12, the digital shifter 14, and the amplitude adjuster 17.

With this arrangement, regardless of the relative positional relationship between the measurement site and the electrodes for applying electrical stimulation, the biomagnetic field measurement device 100 in which little variation occurs in the frequency characteristic among the channels of the SQUID sensor array 140 and the occurrence of the out-of-lock is suppressed can be achieved. In other words, regardless of the location of the electrode to which the electrical stimulation is applied, the loop gain can be finely adjusted, and the biomagnetic field measurement device 100 can be operated stably with respect to any measurement sites, by suppressing the occurrence of the out-of-lock.

For example, even if the electrode for applying electrical stimulation is attached near the measurement site of the magnetic field, the occurrence of the out-of-lock can be suppressed. As a result, in a case where the biomagnetic field induced by electrical stimulation is measured by the SQUID sensor and the digital FLL circuit 10, the biomagnetic field can be measured with high accuracy.

The loop gain can be adjusted for each channel, and thus the SQUID sensor that has been treated as a defective product because the sensor characteristics are out of standard can be saved. Thus, the manufacturing yield of the SQUID sensor can be improved, and the manufacturing cost of the SQUID sensor and the biomagnetic field measurement device 100 can be reduced.

### Second Embodiment

FIG. 14 is a circuit block diagram illustrating an example of a digital FLL circuit provided on the biomagnetic field measurement device according to a second embodiment of the present disclosure. The same components as those of the digital FLL circuit 10 in FIG. 3 are denoted by the same reference numerals, and detailed description thereof will be omitted.

The configuration of the biomagnetic field measurement device 100 on which a digital FLL circuit 20 illustrated in FIG. 14 is provided and the configuration of the biomagnetic field measurement system 1000 including the biomagnetic field measurement device 100 are the same as those illustrated in FIG. 1. Similar to the digital FLL circuit 10 of FIG. 3, the digital FLL circuit 20 is provided for each axis (each channel) of the SQUID sensor, and is provided on the control board provided in the FLL circuit unit 110 of FIG. 1. The digital FLL circuit 20 is an example of an FLL unit, and the SQUID sensor and the digital FLL circuit 20 are examples of a measurement device.

In the digital FLL circuit 20, the amplifier 12 and the amplitude adjuster 17 are removed from the digital FLL circuit 10 illustrated in FIG. 3, and a digital multiplier 21 is added. For example, the digital FLL circuit 20 includes the amplifier 11, the AD converter 13, the digital multiplier 21, the digital integrator 15, the DA converter 16, a voltage-to-current converter 18, and the feedback coil 19 that are connected in series.

For example, the digital multiplier 21 and the digital integrator 15 are implemented by the FPGA provided on the control board of the FLL circuit unit 110. The FPGA may be provided in common to the digital FLL circuits 20 provided on the control board. In such a case, the FPGA includes the digital multipliers 21 and the digital integrators 15 for the channels. Note that the control board of the FLL circuit unit 110 may include, instead of the FPGA, an ASIC in which the digital shifter 14 and the digital integrator 15 are implemented, an individual logic component, a general-purpose DSP, or the like.

The amplifier 11 amplifies, at a fixed amplification factor, an output voltage generated by the SQUID sensor in response to the strength of the magnetic field due to the magnetic flux passing through the SQUID sensor, and then outputs the amplified voltage to the AD converter 13 as a voltage signal. The AD converter 13 converts the analog signal from the amplifier 11 into a digital signal (voltage magnitude) and outputs the digital signal to the digital multiplier 21.

The digital multiplier 21 multiplies the multiplier corresponding to a multiplier adjustment value MUL by the digital voltage magnitude (multiplicand) received from the AD converter 13, and outputs the multiplication result (digital value) to the digital integrator 15. The digital integrator 15 integrates the amount of change in the digital value (voltage magnitude) received from the digital multiplier 21, and outputs the resultant integrated data to the DA converter 16 and the data processing device 130. Note that the data processing device 130 generates the multiplier adjustment value MUL instead of generating the gain adjustment value GCNT1, the shift adjustment value SCNT, and the full-scale adjustment value FSCNT in FIG. 3.

For example, the digital multiplier 21 is a fixed-point multiplier that performs multiplication of fixed-point numbers. The number of digits of the fixed-point numbers (integer part and decimal part) used in the digital multiplier 21 is set to such an extent that the loop gain adjustment can be performed with a predetermined accuracy. For example, the predetermined accuracy is equivalent to the adjustment accuracy of the loop gain adjusted by the amplifier 12, the digital shifter 14, and the amplitude adjuster 17 in FIG. 3. Note that, by increasing the number of bits of the multiplier adjustment value MUL, more detailed control of the loop gain than that of the digital FLL circuit 10 can be performed only by the digital multiplier 21.

With one multiplier adjustment value MUL instead of the three values including the gain adjustment values GCNT1, the shift adjustment value SCNT, and the full-scale adjustment value FSCNT, the adjustment accuracy of the loop gain equal to or higher than that of the digital FLL circuit 10 can be obtained. Therefore, the control of the digital FLL circuit 20 by the data processing device 130 can be made easier than that in FIG. 3. The digital multiplier 21 is an example of an adjustment device that adjusts the loop gain of the digital FLL circuit 20 according to an adjustment value that can be set from outside.

In a case where the digital multiplier 21, the digital integrator 15, and a storage unit such as a register that stores the multiplier adjustment value MUL are implemented by the FPGA, a signal line that supplies the multiplier adjustment value MUL to the digital multiplier 21 is wired only inside the FPGA and is not wired outside the FPGA. Thus, the number of signal lines provided on the control board in which the FPGA, the amplifier 11, and the like are implemented can be made smaller than the number of signal lines provided on the control board in which the digital FLL circuit 10 of FIG. 3 is implemented. As a result, the cost of the control board can be reduced, and the cost of the biomagnetic field measurement device 100 can also be reduced.

Although the circuit scale of the digital multiplier 21 is larger than that of the digital shifter 14 of FIG. 3, the digital multiplier 21 can be provided on the FPGA employed in FIG. 3 together with the digital integrator 15 by employing the fixed-point multiplier. For example, the digital multiplier 21 is provided on the FPGA together with the digital integrator 15. Thus, the digital FLL circuit 20 can be implemented by a circuit board having the same size as the circuit board in which the digital FLL circuit 10 illustrated in FIG. 3 is implemented.

The DA converter 16 converts the signal waveform data, which is the voltage magnitude (digital signal) integrated by the digital integrator 15, into voltage, and outputs the converted voltage to the voltage-to-current converter 18. The voltage-to-current converter 18 converts the voltage from the DA converter 16 into current, and outputs the converted current to the feedback coil 19.

The digital multiplier 21 can be replaced with a floating-point multiplier if a larger scale FPGA or a dedicated DSP are provided on the control board. In such a case, the adjustment accuracy of the loop gain can be improved as compared with the case where the fixed-point multiplier is used. However, in the case where the floating-point multiplier is used, the calculation takes longer than the case where the fixed-point multiplier is used, it is required to consider the influence on the cycle of the processing of the digital FLL circuit 20 and the phase margin of the FLL. Furthermore, in the case where a large-scale FPGA or a dedicated DSP is used, power consumption also increases, and thus it is required to consider the margin of the power supply device 120 (FIG. 1) and the like.

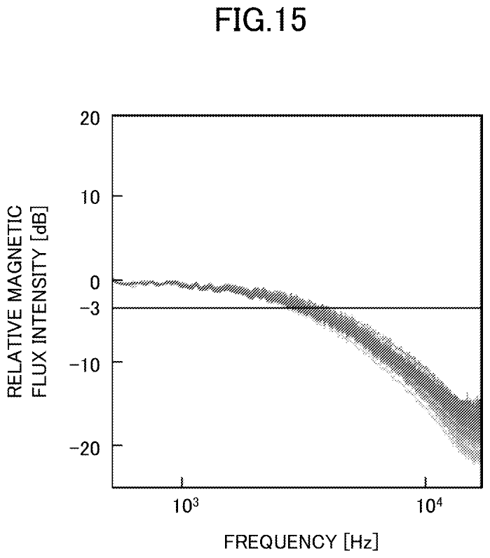

FIG. 15 is a diagram illustrating an example of frequency characteristic after the loop gain is adjusted in the digital FLL circuit 20 of FIG. 14. Detailed description of elements substantially the same as those in FIG. 13 is omitted. Also, in FIG. 15, first, the frequency characteristic before the loop gain is adjusted is obtained as in FIG. 12. Groups are then generated by collecting the channels whose frequency characteristics are close to each other, and the multiplier adjustment value MUL (multiplier) of the digital multiplier 21 is set for each group. With this approach, the frequency characteristic is obtained.

In the example illustrated in FIG. 15, in order to reduce the influence of noise and improve the readability of the cutoff frequency of the FLL as compared with FIG. 13, a magnetic field having a constant intensity in a wide band is applied from outside and the magnetic field is measured. For example, the AD converter 13 outputs 16-bit digital data, and the multiplier adjustment value MUL sent to the digital multiplier 21 is 18-bit fixed-point data in which the integer part is 2 bits and the decimal part is 16 bits.

In FIG. 15, similar to FIG. 12 and FIG. 13, a specific frequency of a specific channel is set as a reference (0 dB), and the cutoff frequency of the FLL circuit is set as −3 dB and illustrated as a line parallel to the horizontal axis (frequency axis). In the frequency characteristic illustrated in FIG. 15, the frequency range at which the curves of the frequency characteristic of each channel intersect with the line of the cutoff frequency is narrower than that in FIG. 13, and it can be seen that the variation in the cutoff frequency is further reduced.

As a result, the occurrence of the out-of-lock can be further suppressed, and a decrease in the measurement accuracy of the biomagnetic field can also be suppressed. Furthermore, as described above, more SQUID sensor that has been treated as a defective product can be saved, and the manufacturing cost of the SQUID sensor and the biomagnetic field measurement device 100 can be further reduced.

As described above, also in the second embodiment, as in the first embodiment, the loop gain of the digital FLL circuit 20 can be adjusted for each channel by making the multiplier used for multiplication by the digital multiplier 21 changeable according to the multiplier adjustment value MUL that can be set from outside, for example.

With this arrangement, regardless of the relative positional relationship between the measurement site and the electrodes for applying electrical stimulation, the biomagnetic field measurement device 100 that suppresses the occurrence of the out-of-lock and operates stably is achieved. For example, even if the electrode for applying electrical stimulation is attached near the measurement site of the magnetic field, the occurrence of the out-of-lock can be suppressed. As a result, in a case where the biomagnetic field induced by electrical stimulation is measured by the SQUID sensor and the digital FLL circuit 20, the biomagnetic field can be measured with high accuracy.

The loop gain can be adjusted for each channel, and thus the SQUID sensor whose characteristics are out of standard can be saved, and the manufacturing yield of the SQUID sensor can be improved. With this arrangement, the manufacturing cost of the SQUID sensor and the biomagnetic field measurement device 100 can be reduced.

Furthermore, in the second embodiment, the adjustment accuracy of the loop gain equal to or higher than that of the digital FLL circuit 10 of FIG. 3 can be obtained by one multiplier adjustment value MUL. Therefore, the control of the digital FLL circuit 20 by the data processing device 130 can be made easier than that in FIG. 3. By adjusting the loop gain by the digital multiplier 21, the frequency characteristic of each channel of the SQUID sensor array 140 can be made uniform with high accuracy as compared with the digital FLL circuit 10 of FIG. 3, and the biomagnetic field can be measured with high accuracy.

### Third Embodiment

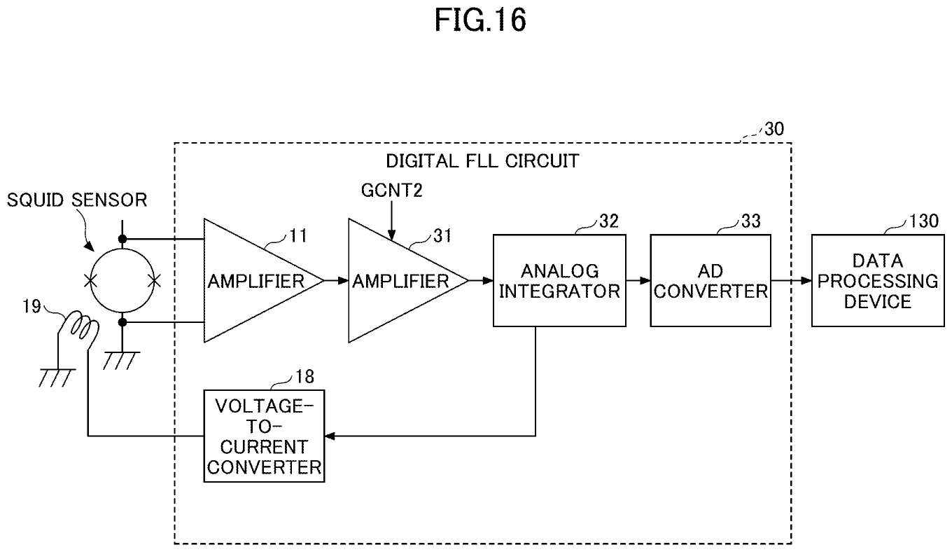

FIG. 16 is a circuit block diagram illustrating an example of an analog FLL circuit provided on the biomagnetic field measurement device according to a third embodiment of the present disclosure. The same components as those of the digital FLL circuit 10 in FIG. 3 are denoted by the same reference numerals, and detailed description thereof will be omitted.

The configuration of the biomagnetic field measurement device 100 on which an analog FLL circuit 30 illustrated in FIG. 16 is provided and the configuration of the biomagnetic field measurement system 1000 including the biomagnetic field measurement device 100 are the same as those illustrated in FIG. 1. Similar to the digital FLL circuit 10 of FIG. 3, the analog FLL circuit 30 is provided for each axis (each channel) of the SQUID sensor, and is provided on the control board provided in the FLL circuit unit 110 of FIG. 1. The analog FLL circuit 30 is an example of an FLL unit, and the SQUID sensor and the analog FLL circuit 30 are examples of a measurement device.

The analog FLL circuit 30 includes an amplifier 31, an analog integrator 32, and an analog-to-digital (AD) converter 33 in addition to the amplifier 11, the voltage-to-current converter 18, and the feedback coil 19 that are the same as those in FIG. 3. For example, the analog FLL circuit 30 includes the amplifier 11, the amplifier 31, the analog integrator 32, the voltage-to-current converter 18, and the feedback coil 19 that are connected in series, and the AD converter 33 connected to the output of the analog integrator 32.

The amplifier 31 can set an amplification factor to any one of the amplification factors in accordance with a gain adjustment value GCNT2, amplifies the voltage received from the amplifier 11 at the set amplification factor, and outputs the amplified voltage to the analog integrator 32 as a voltage signal. The gain adjustment value GCNT2 is stored in, for example, a storage unit such as a register provided on the control board together with the analog FLL circuit 30. The amplifier 31 is an example of an adjustment device that adjusts the loop gain of the analog FLL circuit 30 according to an adjustment value that can be set from outside.

The analog integrator 32 integrates a change in a voltage magnitude obtained by amplifying, by the amplifier 31, a change in voltage of the SQUID sensor from a lock point (or an operating point) that is a starting point of counting of the flux quantum 00, and outputs the integrated voltage magnitude to the voltage-to-current converter 18 and the AD converter 33.

The AD converter 33 samples the voltage magnitude from the analog integrator 32 at a predetermined sampling frequency, and converts the sampled voltage magnitude into a digital voltage magnitude. That is, the AD converter 33 converts, according to the change in the magnetic field, the voltage output from the SQUID sensor into a digital value. The AD converter 33 outputs the digital value obtained by the conversion to the data processing device 130. The voltage-to-current converter 18 converts the voltage from the analog integrator 32 into current, and outputs the converted current to the feedback coil 19.

In the analog FLL circuit 30, the value A indicating the loop gain is proportional to the product of the fixed amplification factor of the amplifier 31 and the variable amplification factor of the amplifier 31, and the value A can be adjusted for each channel of the SQUID sensor. That is, in the analog FLL circuit 30, similar to the digital FLL circuit 10 of FIG. 3, the magnitude of the loop gain of the analog FLL circuit 30 can be adjusted by the amplifier 31.

In the analog FLL circuit 30, the adjustment of the loop gain is performed only by the amplifier 31, and thus the amplifier 31 preferably has a wider adjustment range of the loop gain and a narrower adjustment range than the amplifier 12 of FIG. 3. For example, the number of bits of the gain adjustment value GCNT2 generated by the data processing device 130 is larger than the number of bits of the gain adjustment value GCNT1 in FIG. 3. In other words, by using the amplifier 31 having a wider adjustment range of the loop gain than that of the amplifier 12 of FIG. 3, the loop gain can be adjusted with accuracy equal to or higher than that of the digital FLL circuit 10 by using only the amplifier 31.

Note that one or both of the analog integrator 32 and the voltage-to-current converter 18 may have a variable amplification function. In such a case, the loop gain is the product of the amplification factors of the amplifier 31 and the analog integrator 32, the product of the amplification factors of the amplifier 31 and the voltage-to-current converter 18, or the product of the amplification factors of the amplifier 31, the analog integrator 32, and the voltage-to-current converter 18.

For example, in a case where the adjustment range and the adjustment width of the loop gain are set to be the same as the adjustment range and the adjustment width when the loop gain is adjusted only by the amplifier 31, the total number of bits of the control signal used to control the loop gain is equal to the number of bits of the gain adjustment value GCNT2. In the case where one or both of the analog integrator 32 and the voltage-to-current converter 18 have a variable amplification function, the amplifier 12 of FIG. 3 may be used instead of the amplifier 31.

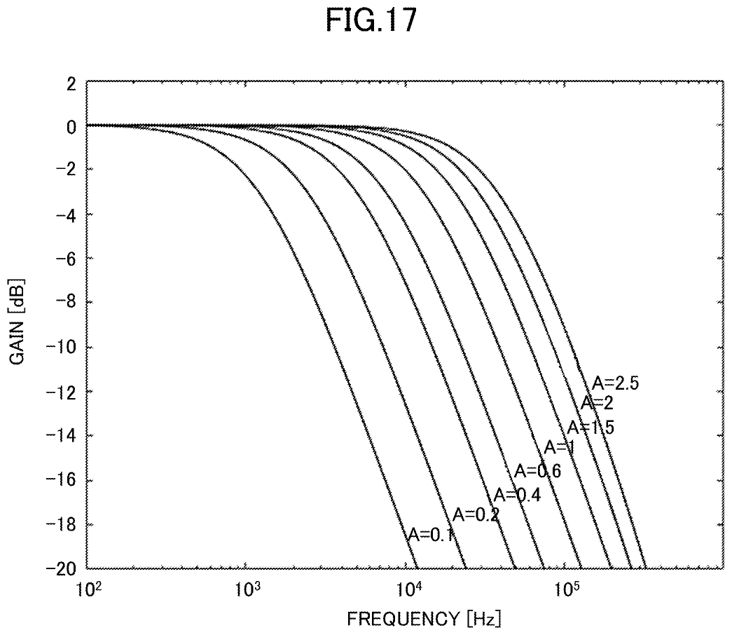

FIG. 17 is a diagram illustrating an example of the closed loop characteristic of the analog FLL circuit 30 illustrated in FIG. 16. In FIG. 17, the magnitude of the output (output voltage) signal relative to the magnetic signal having the frequency indicated on the horizontal axis is indicated as a gain [dB] on the vertical axis. Also, in the analog FLL circuit 30, the closed loop characteristic is similar to the closed loop characteristic of the basic digital FLL circuit of FIG. 7, and it can be seen that the frequency characteristic of the analog FLL circuit 30 can be stably controlled in accordance with the value A with the same behavior as that of the first order low-pass filter.

In the analog FLL circuit 30 of FIG. 16, the value A (loop gain) can be adjusted for each channel of the SQUID sensor by the amplifier 31. For example, the frequency characteristic of the analog FLL circuit 30 can be controlled by appropriately adjusting the A value (loop gain) for each channel of the SQUID sensor in accordance with the position of the SQUID sensor in the SQUID sensor array 140.

As described above, also in the third embodiment, the loop gain of the analog FLL circuit 30 can be adjusted for each channel by making the characteristics of the amplifier 31 changeable according to an adjustment value that can be set from outside, for example, as in the first embodiment and the second embodiment described above.

With this arrangement, regardless of the relative positional relationship between the measurement site and the electrode that applies electrical stimulation, the biomagnetic field measurement device 100 that suppresses the occurrence of the out-of-lock and operates stably can be achieved. For example, even if the electrode for applying electrical stimulation is attached near the measurement site of the magnetic field, the occurrence of the out-of-lock can be suppressed. As a result, in a case where the biomagnetic field induced by electrical stimulation is measured by the SQUID sensor and the FLL unit, the biomagnetic field can be measured with high accuracy.

Because the loop gain can be adjusted for each channel, the SQUID sensor whose characteristics are out of standard can be saved, and the manufacturing yield of the SQUID sensor can be improved. With this arrangement, the manufacturing cost of the SQUID sensor and the biomagnetic field measurement device 100 can be reduced.

The above-described embodiments can be modified as follows. For example, some of the components of the digital FLL circuit 10 of FIG. 3 and the digital FLL circuit 20 of FIG. 14 may be implemented by software. For example, a central processing unit (CPU) or digital signal processing (DSP) may be provided on a control board on which the digital FLL circuit 10 is provided, and one or both of the functions of the digital shifter 14 and the digital integrator 15 may be implemented by software. One or both of the functions of the digital multiplier 21 and the digital integrator 15 of the digital FLL circuit 20 may also be implemented by software.

Furthermore, in the amplifier 11 of FIG. 3, FIG. 14, and FIG. 16, the amplification factor can be changed in accordance with the gain adjustment value. The resistance element of the voltage-to-current converter 18 of FIG. 3, FIG. 14, and FIG. 16 may be a variable resistance element whose resistance value can be changed in accordance with the resistance adjustment value supplied from outside. The loop gain may be adjusted using one or more of functional units (such as amplifiers) whose characteristics can be changed. As described above, in the digital FLL circuit 10 of FIG. 3, the digital FLL circuit 20 of FIG. 14, and the analog FLL circuit 30 of FIG. 16, the loop gain may be adjusted by one or more of functional units whose characteristics can be changed.

The present disclosure has, for example, the following manners.

[1] A biomagnetic field measurement device to measure a biomagnetic field includes a superconducting quantum interference device (SQUID) sensor, and includes a flux locked loop (FLL) unit. The FLL unit includes an adjustment device configured to adjust a loop gain of the FLL unit.

[2] In the biomagnetic field measurement device in [1], the SQUID sensor and the FLL unit are configured to measure an induced magnetic field generated by applying electrical stimulation to a living body by an electrical stimulation input device. The adjustment device is configured to adjust the loop gain of the FLL unit in accordance with an adjustment value settable from outside the biomagnetic field measurement device.

[3] In the biomagnetic field measurement device in [2], the adjustment value is set in accordance with a location or a measurement site of the living body to which the electrical stimulation is applied by the electrical stimulation input device.

[4] The biomagnetic field measurement device in [2] or [3] includes a plurality of measurement devices each including the SQUID sensor and the FLL unit. In any given one of the measurement devices, the adjustment device is configured to set the adjustment value corresponding to a characteristic of the SQUID sensor included in the any given one of the measurement devices.

[5] In the biomagnetic field measurement device in any one of [2] to [4], the FLL unit includes:
an amplifier configured to amplify a voltage signal output from the SQUID sensor;
an analog-to-digital (AD) converter configured to convert the voltage signal amplified by the amplifier into a digital signal;
a digital multiplier configured to multiply a signal value of the digital signal converted by the AD converter by a multiplier corresponding to the adjustment value;
a digital integrator configured to integrate the signal value of the digital signal multiplied by the digital multiplier to generate integrated data;
a digital-to-analog (DA) converter configured to convert the integrated data generated by the digital integrator into a voltage signal;
a voltage-to-current converter configured to convert the voltage signal converted by the DA converter into a current signal; and
a coil configured to feed back the current signal converted by the voltage-to-current converter to the SQUID sensor as a magnetic field, and
the digital multiplier is configured to function as the adjustment device.

[6] In the biomagnetic field measurement device in any one of [2] to [4], the FLL unit includes:
a first amplifier configured to amplify a voltage signal output from the SQUID sensor;
a second amplifier configured to amplify the voltage signal amplified by the first amplifier at any one of a plurality of amplification factors corresponding to a first adjustment value that can be set from outside;
an analog-to-digital (AD) converter configured to convert the voltage signal amplified by the second amplifier into a digital signal;
a digital shifter configured to perform shift processing on a signal value of the digital signal converted by the AD converter by any one of a plurality of shift amounts corresponding to a second adjustment value that can be set from outside;
a digital integrator configured to integrate the signal value of the digital signal subjected to the shift processing by the digital shifter to generate integrated data;
a digital-to-analog (DA) converter configured to convert the integrated data generated by the digital integrator into a voltage signal;
an amplitude adjuster configured to convert a full scale of an output of the DA converter to any one of a plurality of full scales corresponding to a third adjustment value that can be set from outside to adjust to any one of a plurality of amplitudes;
a voltage-to-current converter configured to convert the voltage signal whose voltage magnitude is changed by converting the full scale by the amplitude adjuster into a current signal; and
a coil configured to feed back the current signal converted by the voltage-to-current converter to the SQUID sensor as a magnetic field, and
the second amplifier, the digital shifter, and the amplitude adjuster are configured to function as the adjustment devices.

[7] In the biomagnetic field measurement device in any one of [2] to [4], the FLL unit includes:
a first amplifier configured to amplify a voltage signal output from the SQUID sensor;
a second amplifier configured to amplify the voltage signal amplified by the first amplifier at any one of a plurality of amplification factors corresponding to a first adjustment value that can be set from outside;
an analog-to-digital (AD) converter configured to convert the voltage signal amplified by the second amplifier into a digital signal;
a digital shifter configured to perform shift processing on a signal value of the digital signal converted by the AD converter by any one of a plurality of shift amounts corresponding to a second adjustment value that can be set from outside;
a digital integrator configured to integrate the signal value of the digital signal subjected to the shift processing by the digital shifter to generate integrated data;
a digital-to-analog (DA) converter configured to convert the integrated data generated by the digital integrator into a voltage signal;
a voltage-to-current converter configured to convert the voltage signal converted by the DA converter into a current signal; and
a coil configured to feed back the current signal converted by the voltage-to-current converter to the SQUID sensor as a magnetic field, and
the second amplifier and the digital shifter are configured to function as the adjustment devices.

[8] In the biomagnetic field measurement device in any one of [2] to [4], the FLL unit includes:
a first amplifier configured to amplify a voltage signal output from the SQUID sensor;
an analog-to-digital (AD) converter configured to convert the voltage signal amplified by the first amplifier into a digital signal;
a digital shifter configured to perform shift processing on a signal value of the digital signal converted by the AD converter by any one of a plurality of shift amounts corresponding to a first adjustment value that can be set from outside;
a digital integrator configured to integrate the signal value of the digital signal subjected to the shift processing by the digital shifter to generate integrated data;
a digital-to-analog (DA) converter configured to convert the integrated data generated by the digital integrator into a voltage signal;
an amplitude adjuster configured to convert a full scale of an output of the DA converter to any one of a plurality of full scales corresponding to a second adjustment value that can be set from outside to adjust to any one of a plurality of amplitudes;
a voltage-to-current converter configured to convert the voltage signal whose voltage magnitude is changed by converting the full scale by the amplitude adjuster into a current signal; and
a coil configured to feed back the current signal converted by the voltage-to-current converter to the SQUID sensor as a magnetic field, and
the digital shifter and the amplitude adjuster are configured to function as the adjustment devices.

[9] In the biomagnetic field measurement device in any one of [2] to [4], the FLL unit includes:
a first amplifier configured to amplify a voltage signal output from the SQUID sensor;
a second amplifier configured to amplify the voltage signal amplified by the first amplifier at any one of a plurality of amplification factors corresponding to a first adjustment value that can be set from outside;
an analog-to-digital (AD) converter configured to convert the voltage signal amplified by the second amplifier into a digital signal;
a digital integrator configured to integrate a signal value of the digital signal converted by the AD converter to generate integrated data;
a digital-to-analog (DA) converter configured to convert the integrated data generated by the digital integrator into a voltage signal;
an amplitude adjuster configured to convert a full scale of an output of the DA converter to any one of a plurality of full scales corresponding to a second adjustment value that can be set from outside to adjust to any one of a plurality of amplitudes;
a voltage-to-current converter configured to convert the voltage signal whose voltage magnitude is changed by converting the full scale by the amplitude adjuster into a current signal; and
a coil configured to feed back the current signal converted by the voltage-to-current converter to the SQUID sensor as a magnetic field, and
the second amplifier and the amplitude adjuster are configured to function as the adjustment devices.

[10] In the biomagnetic field measurement device in any one of [2] to [4], the FLL unit includes:
a first amplifier configured to amplify a voltage signal output from the SQUID sensor;
a second amplifier configured to amplify the voltage signal amplified by the first amplifier at any one of a plurality of amplification factors corresponding to the adjustment value that can be set from outside;
an analog-to-digital (AD) converter configured to convert the voltage signal amplified by the second amplifier into a digital signal;
a digital integrator configured to integrate a signal value of the digital signal converted by the AD converter to generate integrated data;
a digital-to-analog (DA) converter configured to convert the integrated data generated by the digital integrator into a voltage signal;
a voltage-to-current converter configured to convert the voltage signal converted by the DA converter into a current signal; and
a coil configured to feed back the current signal converted by the voltage-to-current converter to the SQUID sensor as a magnetic field, and
the second amplifier is configured to function as the adjustment device.

[11] In the biomagnetic field measurement device in any one of [2] to [4], the FLL unit includes:
a first amplifier configured to amplify a voltage signal output from the SQUID sensor;
an analog-to-digital (AD) converter configured to convert the voltage signal amplified by the first amplifier into a digital signal;
a digital shifter configured to perform shift processing on a signal value of the digital signal converted by the AD converter by any one of a plurality of shift amounts corresponding to the adjustment value that can be set from outside;
a digital integrator configured to integrate the signal value of the digital signal subjected to the shift processing by the digital shifter to generate integrated data;
a digital-to-analog (DA) converter configured to convert the integrated data generated by the digital integrator into a voltage signal;
a voltage-to-current converter configured to convert the voltage signal converted by the DA converter into a current signal; and
a coil configured to feed back the current signal converted by the voltage-to-current converter to the SQUID sensor as a magnetic field, and
the digital shifter is configured to function as the adjustment device.

[12] In the biomagnetic field measurement device in any one of [2] to [4], the FLL unit includes:
a first amplifier configured to amplify a voltage signal output from the SQUID sensor;
an analog-to-digital (AD) converter configured to convert the voltage signal amplified by the first amplifier into a digital signal;
a digital integrator configured to integrate a signal value of the digital signal converted by the AD converter to generate integrated data;
a digital-to-analog (DA) converter configured to convert the integrated data generated by the digital integrator into a voltage signal;
an amplitude adjuster configured to convert a full scale of an output of the DA converter to any one of a plurality of full scales corresponding to the adjustment value that can be set from outside to adjust to any one of a plurality of amplitudes;
a voltage-to-current converter configured to convert the voltage signal whose voltage magnitude is changed by converting the full scale by the amplitude adjuster into a current signal; and
a coil configured to feed back the current signal converted by the voltage-to-current converter to the SQUID sensor as a magnetic field, and
the amplitude adjuster is configured to function as the adjustment device.

[13] In the biomagnetic field measurement device in any one of [2] to [4], the FLL unit includes:
a first amplifier configured to amplify a voltage signal output from the SQUID sensor;
a second amplifier configured to amplify the voltage signal amplified by the first amplifier at any one of a plurality of amplification factors corresponding to a first adjustment value that can be set from outside;
an analog integrator configured to integrate the voltage signal amplified by the second amplifier;
a voltage-to-current converter configured to convert the voltage signal integrated by the analog integrator into a current signal; and
a coil configured to feed back the current signal converted by the voltage-to-current converter to the SQUID sensor as a magnetic field, and
the second amplifier is configured to function as the adjustment device.

[14] In the biomagnetic field measurement device in [13], one or both of the analog integrator and the voltage-to-current converter include an amplification function. The amplification function of the analog integrator is a function of changing an amplification factor of a current signal in accordance with an adjustment value that can be set from outside. The amplification function of the voltage-to-current converter is a function of changing a conversion ratio of a current signal with respect to a voltage signal in accordance with an adjustment value that can be set from outside. The adjustment device includes the analog integrator having an amplification function, the voltage-to-current converter having an amplification function, or both the analog integrator having an amplification function and the voltage-to-current converter having an amplification function.

[15] An FLL unit to measure a biomagnetic field in accordance with a voltage signal output from a SQUID sensor includes:
an amplifier configured to amplify a voltage signal output from the SQUID sensor;
an analog-to-digital (AD) converter configured to convert the voltage signal amplified by the amplifier into a digital signal;
a digital multiplier configured to multiply a signal value of the digital signal converted by the AD converter by a multiplier corresponding to the adjustment value that can be set from outside in accordance with the adjustment value;
a digital integrator configured to integrate the signal value of the digital signal multiplied by the digital multiplier to generate integrated data;
a digital-to-analog (DA) converter configured to convert the integrated data generated by the digital integrator into a voltage signal;
a voltage-to-current converter configured to convert the voltage signal converted by the DA converter into a current signal; and
a coil configured to feed back the current signal converted by the voltage-to-current converter to the SQUID sensor as a magnetic field, and
the digital multiplier is configured to function as an adjustment device configured to adjust a loop gain of the FLL unit.

[16] An FLL unit to measure a biomagnetic field in accordance with a voltage signal output from a SQUID sensor includes:
a first amplifier configured to amplify a voltage signal output from the SQUID sensor;
a second amplifier configured to amplify the voltage signal amplified by the first amplifier at any one of a plurality of amplification factors corresponding to a first adjustment value that can be set from outside;
an analog-to-digital (AD) converter configured to convert the voltage signal amplified by the second amplifier into a digital signal;
a digital shifter configured to perform shift processing on a signal value of the digital signal converted by the AD converter by any one of a plurality of shift amounts corresponding to a second adjustment value that can be set from outside;
a digital integrator configured to integrate the signal value of the digital signal subjected to the shift processing by the digital shifter to generate integrated data;
a digital-to-analog (DA) converter configured to convert the integrated data generated by the digital integrator into a voltage signal;
an amplitude adjuster configured to convert a full scale of an output of the DA converter to any one of a plurality of full scales corresponding to a third adjustment value that can be set from outside to adjust to any one of a plurality of amplitudes;
a voltage-to-current converter configured to convert the voltage signal whose voltage magnitude is changed by converting the full scale by the amplitude adjuster into a current signal; and
a coil configured to feed back the current signal converted by the voltage-to-current converter to the SQUID sensor as a magnetic field, and
the second amplifier, the digital shifter, and the amplitude adjuster are configured to function as adjustment devices configured to adjust a loop gain of the FLL unit.

[17] A biomagnetic field measurement system includes:
an electrical stimulation input device configured to generate an induced magnetic field in a living body by applying electrical stimulation to the living body; and
the biomagnetic field measurement device in any one of [1] to [14].

Although the present disclosure has been described above using the embodiments, the present disclosure is by no means limited to the requirements illustrated in the above embodiments. These points can be changed within the scope of the present disclosure, and can be appropriately determined according to the mode of implementation.

When the biomagnetic field is measured using the SQUID sensor and the FLL unit, the biomagnetic field can be accurately measured.

What is claimed is:

1. A biomagnetic field measurement device to measure a biomagnetic field, the biomagnetic field measurement device comprising:
a superconducting quantum interference device (SQUID) sensor; and
a flux locked loop (FLL) unit including an adjustment device configured to adjust a loop gain of the FLL unit.

2. The biomagnetic field measurement device according to claim 1,
wherein the SQUID sensor and the FLL unit are configured to measure an induced magnetic field generated by applying electrical stimulation to a living body by an electrical stimulation input device, and
wherein the adjustment device is configured to adjust the loop gain of the FLL unit in accordance with an adjustment value settable from outside the biomagnetic field measurement device.

3. The biomagnetic field measurement device according to claim 2, wherein the adjustment value is set in accordance with a location or a measurement site of the living body to which the electrical stimulation is applied by the electrical stimulation input device.

4. The biomagnetic field measurement device according to claim 2, further comprising:
a second biomagnetic field measurement device including the SQUID sensor and the FLL unit,
wherein, in each of the biomagnetic field measurement device and the second biomagnetic field measurement device, the adjustment device is configured to set the adjustment value corresponding to a characteristic of the SQUID sensor that is included in a corresponding biomagnetic field measurement device.

5. The biomagnetic field measurement device according to claim 2,
wherein the FLL unit includes:
an amplifier configured to amplify a voltage signal output from the SQUID sensor,
an analog-to-digital (AD)converter configured to convert the voltage signal amplified by the amplifier into a digital signal,
a digital multiplier configured to multiply a signal value of the digital signal converted by the AD converter, by a multiplier corresponding to the adjustment value,
a digital integrator configured to integrate the signal value of the digital signal multiplied by the digital multiplier to generate integrated data,
a digital-to-analog (DA) converter configured to convert the integrated data generated by the digital integrator into a voltage signal,
a voltage-to-current converter configured to convert the voltage signal converted by the DA converter into a current signal, and
a coil configured to feed back the current signal converted by the voltage-to-current converter to the SQUID sensor as a magnetic field, and
wherein the digital multiplier is configured to function as the adjustment device.

6. The biomagnetic field measurement device according to claim 2, wherein the FLL unit includes:
a first amplifier configured to amplify a voltage signal output from the SQUID sensor,
a second amplifier configured to amplify the voltage signal amplified by the first amplifier at any one of a plurality of amplification factors, the plurality of amplification factors corresponding to a first adjustment value to be set from outside,
an analog-to-digital (AD) converter configured to convert the voltage signal amplified by the second amplifier into a digital signal,
a digital shifter configured to perform shift processing on a signal value of the digital signal converted by the AD converter, by any one of a plurality of shift amounts, the plurality of shift amounts corresponding to a second adjustment value to be set from outside,
a digital integrator configured to integrate the signal value of the digital signal subjected to the shift processing by the digital shifter to generate integrated data,
a digital-to-analog (DA) converter configured to convert the integrated data generated by the digital integrator into a voltage signal,
an amplitude adjuster configured to convert a full scale of an output of the DA converter to any one of a plurality of full scales to adjust to any one of a plurality of amplitudes, the plurality of full scales corresponding to a third adjustment value to be set from outside;
a voltage-to-current converter configured to convert the voltage signal whose voltage magnitude is changed by converting the full scale by the amplitude adjuster into a current signal, and
a coil configured to feed back the current signal converted by the voltage-to-current converter to the SQUID sensor as a magnetic field, and
wherein the second amplifier, the digital shifter, and the amplitude adjuster are each configured to function as the adjustment devices.

7. The biomagnetic field measurement device according to claim 2, wherein the FLL unit includes:
a first amplifier configured to amplify a voltage signal output from the SQUID sensor,
a second amplifier configured to amplify the voltage signal amplified by the first amplifier at any one of a plurality of amplification factors, the plurality of amplification factors corresponding to a first adjustment value to be set from outside,
an analog-to-digital (AD) converter configured to convert the voltage signal amplified by the second amplifier into a digital signa,
a digital shifter configured to perform shift processing on a signal value of the digital signal converted by the AD converter by any one of a plurality of shift amounts, the plurality of shift amounts corresponding to a second adjustment value to be set from outside,
a digital integrator configured to integrate the signal value of the digital signal subjected to the shift processing by the digital shifter to generate integrated data,
a digital-to-analog (DA) converter configured to convert the integrated data generated by the digital integrator into a voltage signal,
a voltage-to-current converter configured to convert the voltage signal converted by the DA converter into a current signal, and
a coil configured to feed back the current signal converted by the voltage-to-current converter to the SQUID sensor as a magnetic field, and
wherein the second amplifier and the digital shifter are configured to function as the adjustment devices.

8. The biomagnetic field measurement device according to claim 2, wherein the FLL unit includes:
a first amplifier configured to amplify a voltage signal output from the SQUID sensor,
an analog-to-digital (AD) converter configured to convert the voltage signal amplified by the first amplifier into a digital signal,
a digital shifter configured to perform shift processing on a signal value of the digital signal converted by the AD converter by any one of a plurality of shift amounts, the plurality of shift amounts corresponding to a first adjustment value to be set from outside;
a digital integrator configured to integrate the signal value of the digital signal subjected to the shift processing by the digital shifter to generate integrated data,
a digital-to-analog (DA) converter configured to convert the integrated data generated by the digital integrator into a voltage signal,
an amplitude adjuster configured to convert a full scale of an output of the DA converter to any one of a plurality of full scales to adjust to any one of a plurality of amplitudes, the plurality of full scales corresponding to a second adjustment value to be set from outside,
a voltage-to-current converter configured to convert the voltage signal whose voltage magnitude is changed by converting the full scale by the amplitude adjuster into a current signal, and
a coil configured to feed back the current signal converted by the voltage-to-current converter to the SQUID sensor as a magnetic field, and
wherein the digital shifter and the amplitude adjuster are each configured to function as the adjustment device.

9. The biomagnetic field measurement device according to claim 2, wherein the FLL unit includes:
a first amplifier configured to amplify a voltage signal output from the SQUID sensor,
a second amplifier configured to amplify the voltage signal amplified by the first amplifier at any one of a plurality of amplification factors, the plurality of amplification factors corresponding to a first adjustment value to be set from outside,
an analog-to-digital (AD) converter configured to convert the voltage signal amplified by the second amplifier into a digital signal,
a digital integrator configured to integrate a signal value of the digital signal converted by the AD converter to generate integrated data,
a digital-to-analog (DA) converter configured to convert the integrated data generated by the digital integrator into a voltage signal,
an amplitude adjuster configured to convert a full scale of an output of the DA converter to any one of a plurality of full scales to adjust to any one of a plurality of amplitudes, the plurality of full scales corresponding to a second adjustment value to be set from outside,
a voltage-to-current converter configured to convert the voltage signal whose voltage magnitude is changed by converting the full scale by the amplitude adjuster into a current signal, and
a coil configured to feed back the current signal converted by the voltage-to-current converter to the SQUID sensor as a magnetic field, and
wherein the second amplifier and the amplitude adjuster are each configured to function as the adjustment device.

10. The biomagnetic field measurement device according to claim 2, wherein the FLL unit includes:
a first amplifier configured to amplify a voltage signal output from the SQUID sensor,
a second amplifier configured to amplify the voltage signal amplified by the first amplifier at any one of a plurality of amplification factors, the plurality of amplification factors corresponding to the adjustment value to be set from outside,
an analog-to-digital (AD) converter configured to convert the voltage signal amplified by the second amplifier into a digital signal,
a digital integrator configured to integrate a signal value of the digital signal converted by the AD converter to generate integrated data,
a digital-to-analog (DA) converter configured to convert the integrated data generated by the digital integrator into a voltage signal,
a voltage-to-current converter configured to convert the voltage signal converted by the DA converter into a current signal, and
a coil configured to feed back the current signal converted by the voltage-to-current converter to the SQUID sensor as a magnetic field, and
wherein the second amplifier is configured to function as the adjustment device.

11. The biomagnetic field measurement device according to claim 2, wherein the FLL unit includes:
a first amplifier configured to amplify a voltage signal output from the SQUID sensor,
an analog-to-digital (AD) converter configured to convert the voltage signal amplified by the first amplifier into a digital signal,
a digital shifter configured to perform shift processing on a signal value of the digital signal converted by the AD converter by any one of a plurality of shift amounts, the plurality of shift amounts corresponding to the adjustment value to be set from outside,
a digital integrator configured to integrate the signal value of the digital signal subjected to the shift processing by the digital shifter to generate integrated data,
a digital-to-analog (DA) converter configured to convert the integrated data generated by the digital integrator into a voltage signal,
a voltage-to-current converter configured to convert the voltage signal converted by the DA converter into a current signal, and
a coil configured to feed back the current signal converted by the voltage-to-current converter to the SQUID sensor as a magnetic field, and
wherein the digital shifter is configured to function as the adjustment device.

12. The biomagnetic field measurement device according to claim 2, wherein the FLL unit includes:
a first amplifier configured to amplify a voltage signal output from the SQUID sensor,
an analog-to-digital (AD) converter configured to convert the voltage signal amplified by the first amplifier into a digital signal,
a digital integrator configured to integrate a signal value of the digital signal converted by the AD converter to generate integrated data,
a digital-to-analog (DA) converter configured to convert the integrated data generated by the digital integrator into a voltage signal,
an amplitude adjuster configured to convert a full scale of an output of the DA converter to any one of a plurality of full scales to adjust to any one of a plurality of amplitudes, the plurality of full scales corresponding to the adjustment value to be set from outside;
a voltage-to-current converter configured to convert the voltage signal whose voltage magnitude is changed by converting the full scale by the amplitude adjuster into a current signal, and
a coil configured to feed back the current signal converted by the voltage-to-current converter to the SQUID sensor as a magnetic field, and
wherein the amplitude adjuster is configured to function as the adjustment device.

13. The biomagnetic field measurement device according to claim 2, wherein the FLL unit includes:
a first amplifier configured to amplify a voltage signal output from the SQUID sensor,
a second amplifier configured to amplify the voltage signal amplified by the first amplifier at any one of a plurality of amplification factors, the plurality of amplification factors corresponding to a first adjustment value to be set from outside,
an analog integrator configured to integrate the voltage signal amplified by the second amplifier,
a voltage-to-current converter configured to convert the voltage signal integrated by the analog integrator into a current signal, and
a coil configured to feed back the current signal converted by the voltage-to-current converter to the SQUID sensor as a magnetic field, and
wherein the second amplifier is configured to function as the adjustment device.

14. The biomagnetic field measurement device according to claim 13,
wherein one or both of the analog integrator and the voltage-to-current converter include an amplification function,
wherein the amplification function of the analog integrator is a function of changing an amplification factor of a current signal in accordance with an adjustment value that can be set from outside,
wherein the amplification function of the voltage-to-current converter is a function of changing a conversion ratio of a current signal with respect to a voltage signal in accordance with an adjustment value that can be set from outside, and
wherein the adjustment device includes the analog integrator having a first amplification function, the voltage-to-current converter having a second amplification function, or both the analog integrator having the first amplification function and the voltage-to-current converter having the second amplification function.

15. An full locked loop (FLL) unit for measuring a biomagnetic field in accordance with a voltage signal output from a SQUID sensor, the FLL unit comprising:
an amplifier configured to amplify a voltage signal output from the SQUID sensor;
an analog-to-digital (AD) converter configured to convert the voltage signal amplified by the amplifier into a digital signal;
a digital multiplier configured to multiply, in accordance with an adjustment value, a signal value of the digital signal converted by the AD converter by a multiplier, the multiplier corresponding to the adjustment value to be set from outside;
a digital integrator configured to integrate the signal value of the digital signal multiplied by the digital multiplier to generate integrated data;
a digital-to-analog (DA) converter configured to convert the integrated data generated by the digital integrator into a voltage signal;
a voltage-to-current converter configured to convert the voltage signal converted by the DA converter into a current signal; and
a coil configured to feed back the current signal converted by the voltage-to-current converter to the SQUID sensor as a magnetic field, and
wherein the digital multiplier is configured to function as an adjustment device configured to adjust a loop gain of the FLL unit.

16. An FLL unit for measuring a biomagnetic field according to a voltage signal output from a SQUID sensor, the FLL unit comprising:
a first amplifier configured to amplify a voltage signal output from the SQUID sensor;
a second amplifier configured to amplify the voltage signal amplified by the first amplifier at any one of a plurality of amplification factors corresponding to a first adjustment value that can be set from outside;
an analog-to-digital (AD) converter configured to convert the voltage signal amplified by the second amplifier into a digital signal;
a digital shifter configured to perform shift processing on a signal value of the digital signal converted by the AD converter by any one of a plurality of shift amounts, the plurality of shift amounts corresponding to a second adjustment value to be set from outside;
a digital integrator configured to integrate the signal value of the digital signal subjected to the shift processing by the digital shifter to generate integrated data;
a digital-to-analog (DA) converter configured to convert the integrated data generated by the digital integrator into a voltage signal;
an amplitude adjuster configured to convert a full scale of an output of the DA converter to any one of a plurality of full scales corresponding to a third adjustment value that can be set from outside to adjust to any one of a plurality of amplitudes;
a voltage-to-current converter configured to convert the voltage signal whose voltage magnitude is changed by converting the full scale by the amplitude adjuster into a current signal; and
a coil configured to feed back the current signal converted by the voltage-to-current converter to the SQUID sensor as a magnetic field, and
wherein the second amplifier, the digital shifter, and the amplitude adjuster are each configured to function as the adjustment device configured to adjust a loop gain of the FLL unit.

17. A biomagnetic field measurement system comprising:
an electrical stimulation input device configured to generate an induced magnetic field in a living body by applying electrical stimulation to the living body; and
the biomagnetic field measurement device of claim 1.
